(19) United States
(12) Patent Application Publication
(10) Pub. No.: US 2024/0189225 A1
(43) Pub. Date: Jun. 13, 2024

(54) COMPOSITIONS AND METHODS FOR HARDENING

(71) Applicant: SPI Pharma, Inc., Wilmington, DE (US)

(72) Inventors: Brian Wilson, Grand Haven, MI (US); Greg Simon, Grand Haven, MI (US); Graeme Macleod, Wezembeek Oppem (BE)

(73) Assignee: SPI Pharma, Inc., Wilmington, DE (US)

(21) Appl. No.: 17/998,689

(22) PCT Filed: May 14, 2021
(86) PCT No.: PCT/US2021/032582

§ 371 Date: Nov. 14, 2022

Related U.S. Application Data
(60) Provisional application No. 63/025,362, filed on May 15, 2020.

(51) Int. Cl. A61K 9/00; A61K 9/16; A61K 9/20; A61K 9/28

(52) U.S. Cl. CPC A61K 9/0056; A61K 9/1623; A61K 9/2018; A61K 9/2095; A61K 9/2866; A61K 9/2893

(57) ABSTRACT

The present disclosure relates to, among other things, compositions and methods for improving hardness without using excessive compression forces, thereby preserving compression-sensitive or pressure-sensitive active ingredients. The present disclosure also relates to compositions and methods for preparing post-compression hardening materials having a high tensile strength at low water activity.

### CROSS-REFERENCE TO RELATED APPLICATIONS

This application is an International Application, which claims priority to U.S. Provisional Application No. 63/025,362, filed May 15, 2020, the entirety of which is incorporated herein by reference.

### FIELD

The present disclosure relates to, among other things, compositions and methods for improving hardness without using excessive compression forces, thereby preserving compression-sensitive or pressure-sensitive active ingredients. The present disclosure also relates to compositions and methods for preparing post compression hardening materials having a high tensile strength at low water activity.

### BACKGROUND OF THE INVENTION

Many industry standard binders fail to provide adequate protection for active pharmaceutical ingredients (APIs), probiotics, and other materials that have sensitivities to moisture, temperature, or pressure, one or more of which can be needed by industry standard binders for increasing tablet hardness over time. Accordingly, industry standard binders exhibit undesirable properties; including diminished tabletability at low active water conditions, poor tablet tensile strength at low compression forces leading to higher compression forces needed, or some products require specialized curing processes using humidity or heat to establish hard tablets. There exists a need for binders that preserve active ingredient functionality and yield a high tablet tensile strength using reduced pressure, low moisture environment.

### BRIEF DESCRIPTION OF THE DRAWINGS

The presently disclosed embodiments will be further explained with reference to the attached drawings. The drawings generally illustrate the principles of the presently disclosed embodiments.

FIGS. 1A-B illustrate (A) bar charts comparing of the compression force (in kilonewtons; kN), initial hardness (in kilopond; kP), and hardness after 24 hours, and (B) a bar chart comparing the percent change in hardness of dosage forms prepared using commercially available materials (PH102, Mannogem XL, Compressol SM, and an experimental Buchi Spray Dry Sample).

FIGS. 2A-B illustrate (A) bar charts comparing of the compression force, initial hardness, and hardness after 24 hours, and (B) a bar chart comparing the percent change in hardness of dosage forms prepared using post-compression hardening compositions comprising varying concentrations of sorbitol or maltitol.

FIGS. 3A-B illustrate (A) bar charts comparing of the compression force, initial hardness, and hardness after 24 hours, and (B) a bar chart comparing the percent change in hardness of dosage forms prepared using post-compression hardening compositions comprising varying concentrations of sorbitol or maltitol, as well as compositions comprising varying mixtures of sorbitol and maltitol.

FIGS. 4A-B illustrate (A) bar charts comparing of the percentage of colony forming units (CFU) preserved after forming a dosage composition using a compression force between about 7.7 kP and 8.8 kP, and (B) the percentage of CFUs preserved after forming a dosage form normalized to compression force used to prepare the dosage form using post-compression hardening compositions comprising varying concentrations of sorbitol or maltitol, as well as compositions comprising varying mixtures of sorbitol and maltitol.

FIG. 5 illustrates the hardness of dosage forms prepared using post-compression hardening compositions comprising varying concentrations of sorbitol or maltitol, as well as compositions comprising varying mixtures of sorbitol and maltitol, normalized to compression force used to prepare the dosage form.

FIG. 6 illustrates the dissolution of Griseofulvin from tablets made from directly compressible lactose and from 80:10:10 mannitol:sorbitol:maltitol co-spray dried process.

FIG. 7 illustrates the hardness values at t0 and t 24 for the formulations described above at various compression forces.

FIG. 8 illustrates the dissolution release of acyclovir from tablets made with different binder systems.

FIG. 9 illustrates compressed MUPS tablets of the present disclosure.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

### Overview

In certain embodiments, the present disclosure pertains generally to directly compressible binders in the delivery of APIs, probiotics, and other pressure and/or moisture sensitive materials. More specifically, the present disclosure relates to a polyol based co-processed material that provides high tabletability in low active water conditions. The co-processed material includes both a high initial tablet hardness (tensile strength) per compression force upon compression and a great increase in tablet hardness after a holding time, without the need for activation by moisture or temperature. The flexibility and simplicity in use of the co-processed material in certain embodiments of the present disclosure has superior retention in colony forming units of a probiotic or in highly engineered nutraceutical and pharmaceutical active ingredients.

Certain embodiments of the present disclosure possess numerous benefits and advantages over known tablet binders. Most notably, the binder in certain embodiments of the present disclosure does not demonstrate a diminished tabletability at low active water conditions, like that observed in most industry standard binders. Certain embodiments of the present disclosure described herein utilizes a distinct mechanism of increasing hardness over time that does not rely on activation through moisture or temperature but instead by direct compression alone. By using this alternative curing mechanism, certain embodiments of the present disclosure provide protection for API, probiotics, and other materials that have sensitivities to moisture, temperature, or pressure which is the foundation of other reported curing methods.

Certain embodiments of the present disclosure avoid curing that causes further consolidation of the material, like moisture activated hardening observed for sugars and polymers. This results in a naturally lower decrease in the disintegration time per hardness increase for the present disclosure over other curing methods. The co-processed materials of the present disclosure further provide the ability to improve disintegration and dissolution of API by retaining tablet porosity during the increase in hardness, relative to materials compressed to this reach this hardness.

There are benefits in improving tooling costs and in the costs of ongoing maintenance. A practical example of the flexibility possessed by certain embodiments of the present disclosure resides in its ability to yield a high tablet tensile strength using a low compression force over a conventional tablet binder. For instance, user specifications may demand that the pressure experienced within the consolidation of material by direct compression to remain low enough to ensure the integrity of either a living organism or a functional coating used in taste masking or directed delivery.

Similarly, the mechanical features of the direct compression binders of the present disclosure allow them to be utilized in several pharmaceutical and nutraceutical manufacturing processes where a carrier is processed through consolidation to yield a robust yet functional end product. As previously discussed, the simplicity of its usage liberates it from the specialized additional processing, like humidity or temperature, used in creating a curing affect in other binder systems.

It can thus be seen that certain embodiments of the present disclosure, which provides a novel solution which successfully reduces losses of active ingredient functionality by reducing pressure within a low moisture environment.

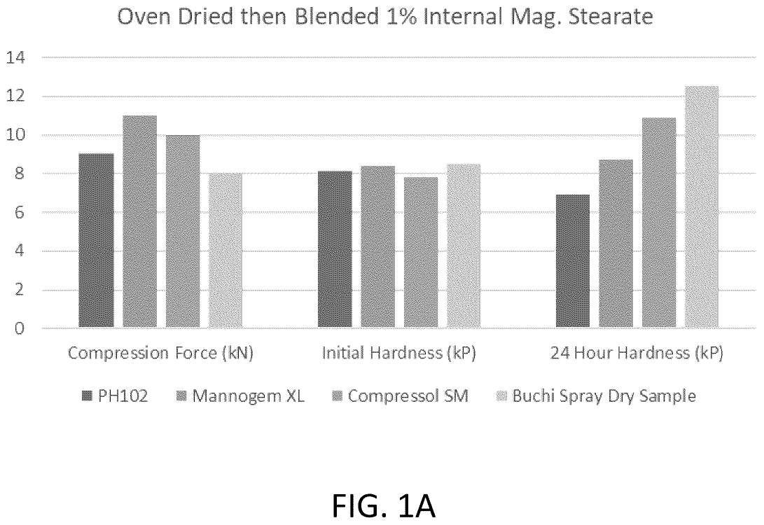

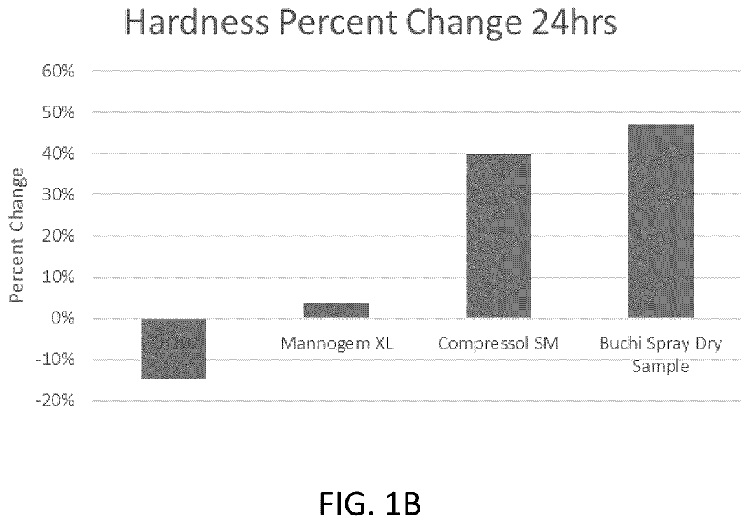

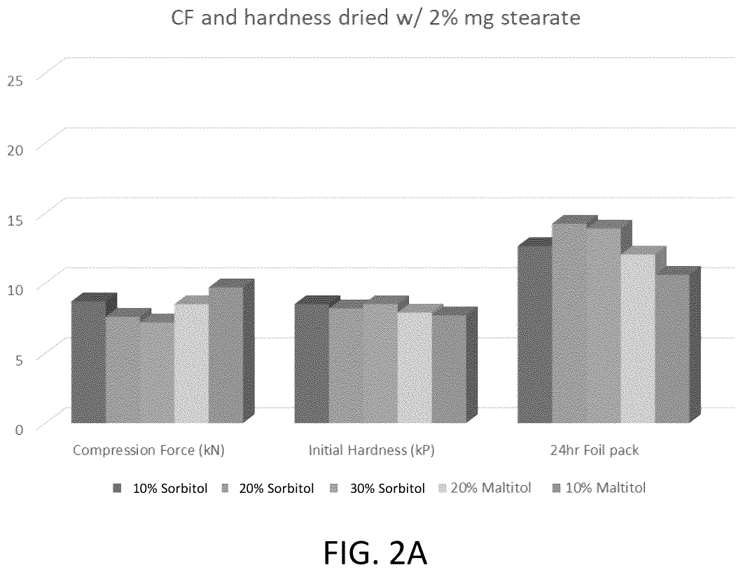

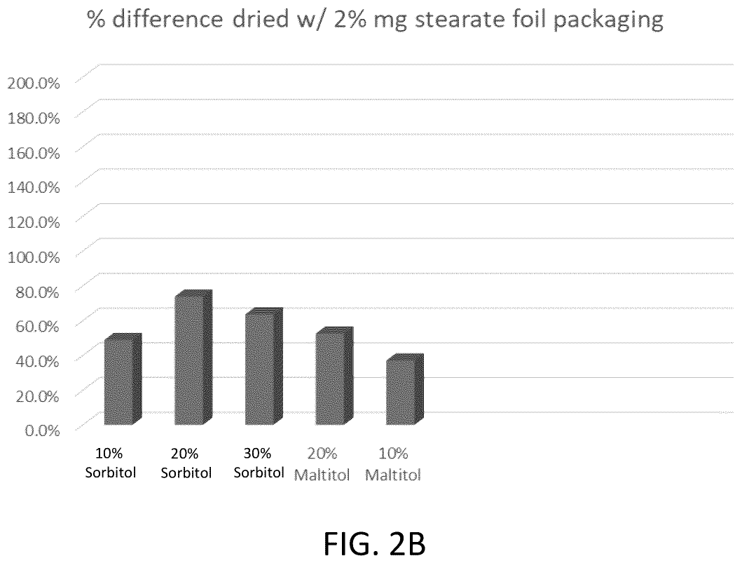

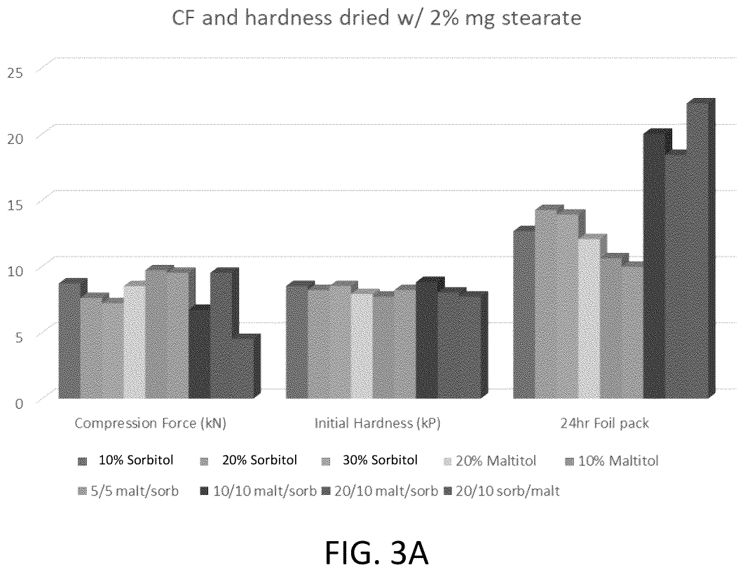

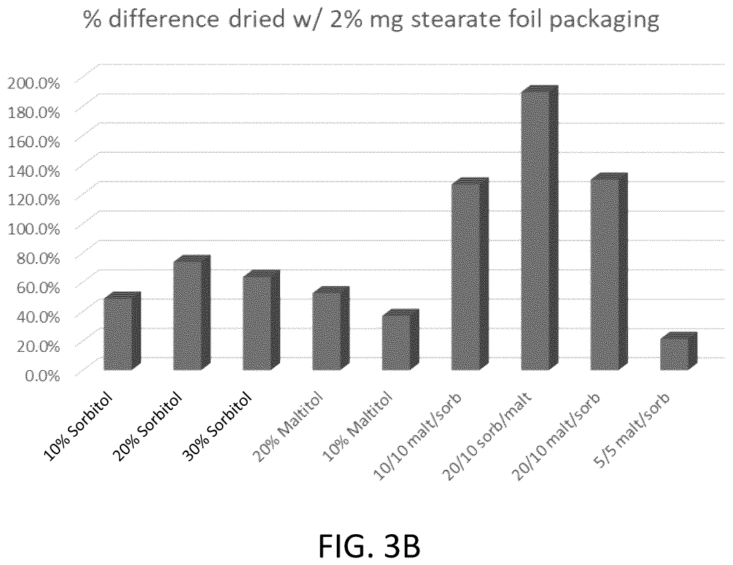

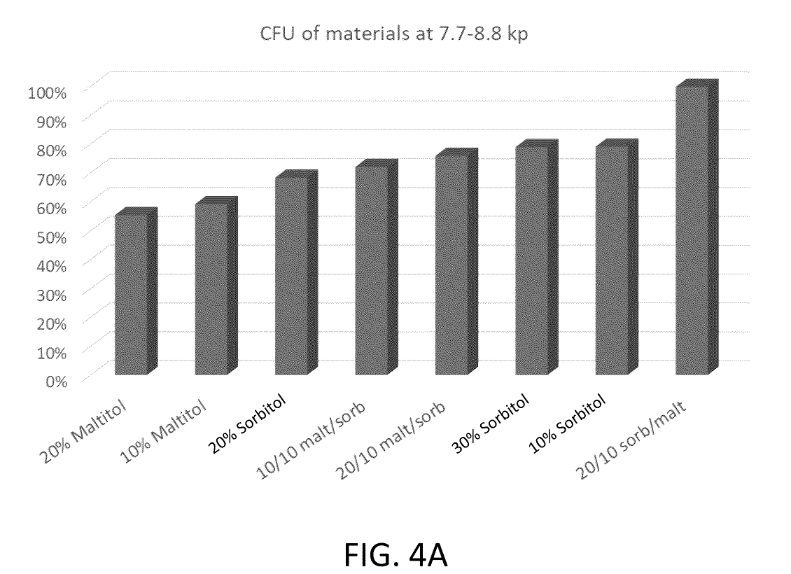

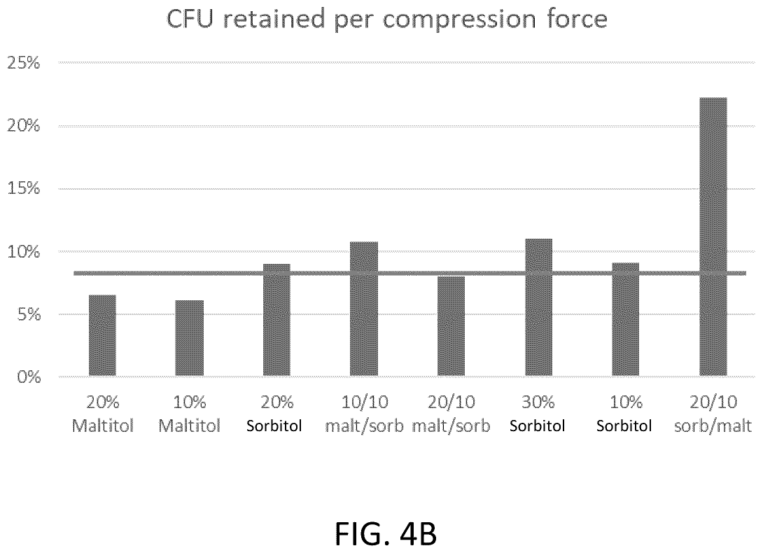

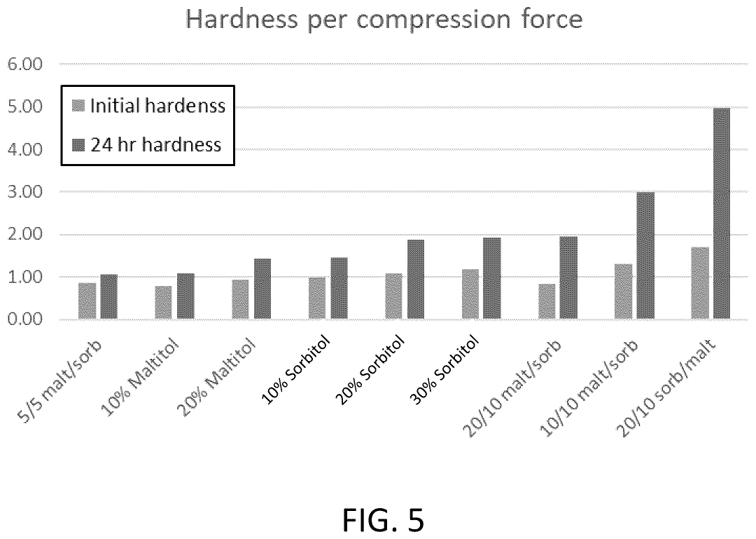

Existing commercially available binders or formulations granulated to enhance tabletability typically require an applied compression force of greater than 10 kN to achieve a resultant tablet that meets the requirements of robustness. For formulations that are higher in active concentration the compression force needed can be considerably higher e.g. 20 kN or above. Robustness of tablets is typically expressed in terms of friability and hardness of the resultant tablet. Tablet hardness is normally expressed in units of N or Kiloponds (Kp). 1 Kilopond is equivalent to 9.81 N. Typical values for hardness of tablets required depend on the size and shape of the tablet and the end use (e.g., chewables and ODT's may have higher friability and still be acceptable as they are typically not coated downstream). Although it is hard to generalize a reasonable rule of thumb for a target hardness would be 80-120 N or 8-10 Kps. Formulators may use Tensile Strength as a means of targeting tablet hardness as this removes the need to consider shape and size effects. A Tensile Strength of at least 1.5 MPa can be targeted. For an 11.3 mm tablet of around 3.7 mm thickness this would equate to a hardness of around 100 N. If one uses a hardness in Kp, to compression Force (in Kn) ratio, a ratio of greater than 1 would be seen to give tablets of suitable robustness. In addition to friability hardness values also need to be considered. Although monograph limits for friability are less than 1% many formulators target friabilities less than 0.5% or lower. The reason for this is that the resultant tablets need to be resistant to further downstream processing such as coating and packaging. Existing commercially available binders exhibit limited post-compression hardening over time. As shown in FIGS. 1A-B, under compression forces between about 8-11 kN, dosage forms prepared using various commercially available binders exhibited limited increases in hardness after 24 hours of storage. In fact, PH102, a microcrystalline cellulose binder used to provide cushioning effects against pressure exhibited an approximately 15% decrease in dosage hardness after 24 hours of storage. Dosage forms prepared using other binders, such as Mannogem XL, Compressol SM, and Sample exhibited between about 5% and 50% increases in hardness. Similarly, as shown for example in FIGS. 2A-B and Table 1, under compression forces between about 5-10 kN, dosage forms prepared using various concentrations of either sorbitol or maltitol only also exhibited limited increases in hardness after 24 hours of storage. Dosage forms prepared using 10%-30% sorbitol or 10%-20% maltitol exhibited between about 30% and 70% increases in hardness. In certain embodiments, an increase in post-compression hardness is observed in at most 0.5 hours, at most 1 hour, at most 2 hours, at most 3 hours, at most 4 hours, at most 5 hours, at most 6 hours, at most 7 hours, at most 8 hours, at most 9 hours, at most 10 hours, at most 11 hours, or at most 12 hours after compaction or compression. In certain embodiments, an increase in post-compression hardness is no longer observed (e.g., maximum post-compression hardness is obtained) after 0.5 hours, after 1 hour, after 2 hours, after 3 hours, after 4 hours, after 5 hours, after 6 hours, after 7 hours, after 8 hours, after 9 hours, after 10 hours, after 11 hours, or after 12 hours after compaction or compression. In certain embodiments, maximum post-compression hardness is obtained within 4 hours after compression or compaction. In certain embodiments, maximum post-compression hardness is obtained within 6 hours after compression or compaction. In certain embodiments, maximum post-compression hardness is obtained within 8 hours after compression or compaction. In certain embodiments, maximum post-compression hardness is obtained between 4 hours and about 8 hours after compression or compaction.

The present disclosure relates to compositions and methods for post-compression or post-compaction hardening, thereby enabling the production of dosage forms having a hardness that would otherwise not be achievable without greater compression force. In other words, a dosage form produced without the post-compression hardening compositions of the present disclosure would require greater compression forces to yield the same hardness as a dosage form produced with the post-compression hardening compositions of the present disclosure, or alternatively not exhibit sufficient hardness to give a viable robust tablet. By utilizing post-compression hardening to drive hardness of the dosage form instead of utilizing greater compression forces, the porosity (and therefore the disintegration time) of the dosage form is maintained. Greater compression forces can result in a dosage form that is less porous or denser. In certain embodiments, the use of a composition with post-compression hardening from the present disclosure allows the production of a dosage form having the same porosity, but greater hardness as compared to a dosage form produced without the post-compression hardening compositions of the present disclosure. In certain embodiments, the use of post-compression hardening compositions of the present disclosure allow for the production of a dosage form having the same hardness, but greater porosity as compared to a dosage form produced without the post-compression hardening compositions of the present disclosure. As disintegration time is a function of porosity, in certain embodiments the dosage forms prepared using the post-compression hardening compositions of the present disclosure retain or have a greater disintegration time as compared to a dosage form produced without the post-compression hardening compositions of the present disclosure. Similarly, as dissolution time is a function of porosity, in certain embodiments the dosage forms prepared using the post-compression hardening compositions of the present disclosure retain or have a greater dissolution time as compared to a dosage form produced without the post-compression hardening compositions of the present disclosure.

The present disclosure also relates to compositions and methods that can be used to preserve compression-sensitive or pressure-sensitive active ingredients in different dosage forms (e.g., granules, tablets, wafers, compacts or ribbons). By utilizing post-compression hardening to drive hardness of the dosage form post-compression, the amount of compression force needed to manufacture the dosage form can be reduced, thereby protecting compression-sensitive or pressure-sensitive active ingredients from excess force that can cause degradation. Use of post-compression hardening to drive durability and hardness of the dosage form post-compression allows for the manufacture of high tensile strength materials with low compression forces and under low water activity, which is an uncommon characteristic in commercially available excipients.

The present disclosure also relates to a direct compression binder which provides with a unique approach of generating hard compacts using the tabletability of the material and a post compression relaxation mechanism. Although the results can be affected by moisture and temperature, the mechanism of curing is independent of their influence for activation. The combinations of ingredients within the co-processed materials in conjunction with manufacturing conditions yields the materials superior performance. The activation of the curing effect occurs post compression at low active water conditions. The lower compression force needed to generate tablets of acceptable hardness and friability allows for both the preservation of activity in pressure sensitive materials as well as the retention of lower disintegration times due to the lower compression forces used.

### Definitions

As used herein, the term “hardness” means the property of a composition (e.g., a granule, dosage form, tablet, wafer, ribbon, or the like) enables it to resist deformation, usually by penetration. However, the term hardness may also refer to resistance to bending, scratching, abrasion or cutting. One method to achieve a hardness value is to measure the depth or area of an indentation left by an indenter of a specific force applied for a specific time. Hardness may be measured at any point and after any known treatment such as, for example, before and after storage of a dosage form for a specified hold time. In certain embodiments, hardness of a dosage form (e.g., a tablet) can be determined using compression. The dosage form can be placed on the holder of the Schleuniger hardness tester (e.g., between two jaws that crush the tablet), and a force is applied on the dosage form with a constant speed. The force applied to the tablet is measured and it is detected when the dosage form fractures.

The term “compression force” can refer to a force applied to an object (e.g., a dosage form) that causes that object to press together or occupy less space. As used herein, compression force can refer to the force used to compress a composition into a desired dosage form such as a tablet, wafer, or ribbon. A compression force is a force that is applied in the opposite direction of a force that would stretch or strain an object. For example, pressing on an object would apply a compression force. As used herein, the term “compression force” can refer to a force over a given area. Excess pressure can cause adverse effects to sensitive active ingredients; compositions and methods of the present disclosure can reduce or eliminate these adverse effects.

The term “water activity” (Aw), as used herein, can be measured at 25° C. and 1 Atmosphere. The term is a quantitative term describing the availability of water for any chemical interaction. In pharmaceutics it is commonly used in sorption isotherms which describe the relation between water content of a product and the corresponding relative humidity (RH) of the air in equilibrium with the product at that water content. The equilibrium RH is directly correlated to the water activity, that is: Water Activity=RH/100. Low water activities in dosage forms are generally advantageous because they are associated with a lower tendency towards microbial growth and a lower tendency towards hydrolytic degradation of moisture-sensitive active pharmaceutical ingredients. Also, high water activities can negatively impact physicochemical properties such as appearance, hardness, and/or dissolution.

The term “loss-on-drying” refers to an evaporated amount of water, solvent and/or volatile materials in a sample, expressed as a percentage (%) based on the weight of sample before drying when the sample is dried under heating condition. Water content can be determined based on water activity, and water content is defined as the content of water determined by the Karl-Fischer method, implying that this water content includes, for example, the amount of crystal water of the ingredients of the tablet. The present disclosure provides compositions and methods for post-compression hardening materials having high tensile strength at low water activity.

The term “colony-forming unit” (CFU) refers to a unit that is used to estimate the number of bacteria, yeast or fungal cells in a sample, that can be for example a cell culture, a feed additive or a feed composition. Although generally used when referring to viable bacteria, the term colony forming unit, or CFU can also be defined as a single non-viable or non-culturable bacterial cell.

The term “compression-sensitive” or “pressure-sensitive” can refer to an active ingredient that deteriorates when exposed to excessive amounts of pressure by compression or granulation or general consolidation. In pharmaceutics, compression-sensitive or pressure-sensitive active ingredients, for example, can refer to probiotics which can become non-viable when compressed into a dosage form such as a tablet. Compression-sensitive or pressure-sensitive active ingredients can also refer to, for example, coated active pharmaceutical ingredients and/or shear sensitive crystalline materials. The present disclosure provides compositions and methods for creating materials capable of maximizing post-compression hardening to have improved hardness at low compression forces.

Disintegration is a process with which substances are broken down into tiny fragments to improve their solubility. The term “disintegration time” generally refers to the time it takes for a dosage form to break down into fragments in a standard test system. For example, the disintegration time can be determined by placing a dosage form into a solution at given temperature and pressure (e.g., distilled water at standard temperature and pressure), and detecting the time for the dosage form to break into particles of less than a given size without stirring.

Dissolution is a process through which solid, gaseous or liquid substances dissolve in a solvent to produce a solution and can be used to determine how soluble a drug is in the body. The term “dissolution time” generally refers to the time it takes for a dosage form to dissolve in a solvent and can be measured using a dissolution test. A dissolution test can be used to detect changes in physical properties of drugs, particularly the active pharmaceutical ingredient (API). Poor solubility can reduce the dissolution rate, and ultimately the bioavailability of the API in the body.

The term “co-processed” can refer to the processing of two or more polyols together to form a homogenous mixture.

“Carrier” or “vehicle” as used herein refer to carrier materials suitable for drug administration. Carriers and vehicles useful herein include any such materials known in the art, e.g., any liquid, gel, solvent, liquid diluent, solubilizer, surfactant, or the like, which is nontoxic, and which does not interact with other components of the composition in a deleterious manner.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The terms “active pharmaceutical ingredient”, “active ingredient”, “single active”, or “API” may refer to an ingredient that is biologically active. In some cases, the pharmaceutical sample contains one API. In some cases, the pharmaceutical sample contains more than one API.

The term “probiotic” refers to microorganisms, which when administered in adequate amounts confer health benefits on the host.

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of various embodiments of the present disclosure is contemplated. Additional active pharmaceutical ingredients, such as other drugs, can also be incorporated into the described compositions and methods.

The term “pharmaceutically acceptable excipient” is intended to include vehicles and carriers capable of being co-administered with a compound to facilitate the performance of its intended function. The use of such media for pharmaceutically active substances is well known in the art. Examples of such vehicles and carriers include solutions, solvents, dispersion media, delay agents, emulsions and the like. Any other conventional carrier suitable for use with the multi-binding compounds also falls within the scope of the present disclosure.

As used herein, the term “a”, “an”, or “the” generally is construed to cover both the singular and the plural forms.

The terms “about” and “approximately” mean within a statistically meaningful range of a value. Such a range can be within an order of magnitude, preferably within 50%, more preferably within 20%, more preferably still within 10%, and even more preferably within 5% of a given value or range. The allowable variation encompassed by the terms “about” or “approximately” depends on the particular system under study, and can be readily appreciated by one of ordinary skill in the art. Moreover, as used herein, the terms “about” and “approximately” mean that compositions, amounts, formulations, parameters, shapes and other quantities and characteristics are not and need not be exact, but can be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

The term “substantially” as used herein can refer to a majority of, or mostly, as in at least about 50%, 60%, 70%, 80%, 90%, 95%, 96%, 97%, 98%, 99%, 99.5%, 99.9%, 99.99%, or at least about 99.999% or more.

The transitional terms “comprising,” “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed embodiments. All compositions, methods, and kits described herein can, in alternate embodiments, be more specifically defined by any of the transitional terms “comprising,” “consisting essentially of,” and “consisting of.”

Compositions of the present disclosure can be suitable for humans (e.g., edible for a human subject with minimal to no adverse side effects, or legally suitable and approved as nourishment for humans). A subject treated by any of the methods or compositions described herein can be a human of any age and can be an adult, infant or child.

Compositions of the present disclosure can be suitable for animals or suitable for veterinary use (e.g., edible for a non-human subject with minimal to no adverse side effects, or legally suitable and approved as nourishment for non-humans). Any of the compositions disclosed herein can be administered to a non-human subject, such as a laboratory or farm animal. Non-limiting examples of a non-human subject include laboratory or research animals, a dog, a goat, a guinea pig, a hamster, a mouse, a pig, a non-human primate (e.g., a gorilla, an ape, an orangutan, a lemur, or a baboon), a rat, a sheep, or a cow.

### Formulations

Post-compression hardening compositions of the present disclosure generally comprise two or more polyols co-processed to form a homogeneous material, wherein the two or more polyols comprises a first polyol present in an amount of about 5 wt % to about 25 wt % of the total weight post-compression hardening composition, and a second polyol present in an amount of 5 wt % to 25 wt % of the total weight of the post-compression hardening composition. In certain embodiments, the two or more polyols can comprise a third main polyol (e.g., mannitol) in an amount of about 50 wt % to about 90 wt % of the total weight of the post-compression hardening composition. In certain embodiments, the two or more polyols can comprise a third and a fourth main polyol in an amount of about 50 wt % to about 90 wt % of the total weight of the post-compression hardening composition. In certain embodiments, the two or more polyols can comprise a third, a fourth, and a fifth main polyol in an amount of about 50 wt % to about 90 wt % of the total weight of the post-compression hardening composition.

Dosage forms (e.g., compositions comprising the post-compression hardening composition and at least one active ingredient) prepared using the post-compression hardening compositions of the present disclosure generally comprise two or more polyols co-processed to form a homogeneous material, wherein the two or more polyols comprises a first polyol present in an amount of about 5 wt % to about 25 wt % of the total weight of the two or more polyols, and a second polyol present in an amount of 5 wt % to 25 wt % of the total weight of the two or more polyols, and one or more compression-sensitive or pressure-sensitive active ingredients, wherein a hardness of the solid dosage form per compression force used to form the solid dosage form is at least about 2.0 after less than about 24 hours of storage (e.g., in desiccated conditions, or in the absence of moisture and/or heat). In certain embodiments, the two or more polyols can comprise a third main polyol (e.g., mannitol) in an amount of about 50 wt % to about 90 wt % of the total weight of the two or more polyols. In certain embodiments, the two or more polyols can comprise a third and a fourth main polyol in an amount of about 50 wt % to about 90 wt % of the total weight of the two or more polyols. In certain embodiments, the two or more polyols can comprise a third, a fourth, and a fifth main polyol in an amount of about 50 wt % to about 90 wt % of the total weight of the two or more polyols.

In some respects, methods of the present disclosure can be used in the manufacture of dosage forms having minimal to no water activity. In certain embodiments, the composition (e.g., a dosage form comprising both the post-compression hardening excipients and the active ingredient) comprises low active water. In certain embodiments, the active water of the compositions is less than about 0.7, less than about 0.6, less than about 0.5, less than about 0.4, less than about 0.3, less than about 0.2, less than about 0.1, less than about 0.05, or less than about 0.01.

In certain embodiments, post-compression hardening is used to increase hardness of a dosage form over a period of storage time (e.g., in desiccated conditions, or in the absence of moisture and/or heat). The relationship between the hardness of the dosage form and the compression force used to manufacture the dosage form (e.g., the hardness per compression force) both before and after storage can be used as a measure of the post-compression hardening observed during storage. In certain embodiments, initial hardness per compression force of a dosage form of the present disclosure (e.g., prior to storage, and with or without an active pharmaceutical ingredient) is at most about 0.001 kilopond (kP) per kilonewton (kN), at most about 0.002 kP/kN, at most about 0.003 kP/kN, at most about 0.004 kP/kN, at most about 0.005 kP/kN, at most about 0.0075 kP/kN, at most about 0.01 kP/kN, at most about 0.1 kP/kN, at most about 0.25 kP/kN, at most about 0.5 kP/kN, at most about 1.0 kP/kN, at most about 1.5 kP/kN, at most about 2.0 kP/kN, or at most about 2.5 kP/kN. In certain embodiments, hardness per compression force of a dosage form of the present disclosure after post-compression hardening (e.g., after storage) is at least about 2.0 kP/kN, at least about 2.5 kP/kN, at least about 3.0 kP/kN, at least about 3.5 kP/kN, at least about 4.0 kP/kN, at least about 4.5 kP/kN, at least about 5.0 kP/kN, at least about 5.5 kP/kN, at least about 6.0 kP/kN, at least about 6.5 kP/kN, at least about 7.0 kP/kN, at least about 7.5 kP/kN, or at least about 10 kP/kN after the period of storage time. In certain embodiments, the period of storage time is about 6 hours, about 12 hours, about 16 hours, about 24 hours, about 36 hours, about 48 hours, or greater than about 48 hours.

In certain embodiments, post-compression hardening is used to increase hardness of a dosage form over a period of storage time (e.g., in desiccated conditions, or in the absence of moisture and/or heat). In certain embodiments, the initial hardness of a dosage form of the present disclosure (e.g., prior to storage, and with or without an active pharmaceutical ingredient) is at most about 1.0 kilopond (kP), at most about 1.5 kP, at most about 2.0 kP, at most about 2.5 kP, at most about 3.0 kP, at most about 3.5 kP, at most about 4.0 kP, at most about 4.5 kP, at most about 5.0 kP, at most about 5.5 kP, at most about 6.0 kP, at most about 6.5 kP, at most about 7.0 kP, at most about 7.5 kP, at most about 8.0 kP, at most about 8.5 kP, at most about 9.0 kP, at most about 9.5 kP, at most about 10.0 kP, at most about 11 kP, at most about 12 kP, at most about 13 kP, at most about 14 kP, or at most about 15 kP. In certain embodiments, hardness of a dosage form of the present disclosure after post-compression hardening (e.g., after storage) is at least about 0.001 kP, at least about 0.005 kP, at least about 0.01 kP, at least about 0.05 kP, at least about 0.1 kP, at least about 0.25 kP, at least about 0.5 kP, at least about 1 kP, at least about 2 kP, at least about 3 kP, at least about 4 kP, at least about 5 kP, at least about 6 kP, at least about 7 kP, at least about 8 kP, at least about 9 kP, at least about 10 kP, at least about 11 kP, at least about 12 kP, at least about 13 kP, at least about 14 kP, at least about 15 kP, at least about 16 kP, at least about 17 kP, at least about 18 kP, at least about 19 kP, at least about 20 kP, at least about 21 kP, at least about 22 kP, at least about 23 kP, at least about 24 kP, at least about 25 kP, at least about 30 kP, at least about 35 kP, or at least about 40 kP after the period of storage time. In certain embodiments, the period of storage time is about 6 hours, about 12 hours, about 16 hours, about 24 hours, about 36 hours, about 48 hours, or greater than about 48 hours.

In certain embodiments, post-compression hardening is used to increase hardness of a dosage form over a period of storage time (e.g., in desiccated conditions, or in the absence of moisture and/or heat). In certain embodiments, the hardness of a dosage form of the present disclosure (with or without an active pharmaceutical ingredient) after post-compression hardening (e.g., after storage) is at least about 25%, 30%, 35%, 40%, 45%, 50%, 55%, 60%, 65%, 70%, 75%, 80%, 85%, 90%, 95%, 100%, 110%, 120%, 130%, 140%, 150%, 175%, 200%, 225%, 250%, 300%, 350%, 400%, 450%, or 500% greater than the initial hardness of the dosage form (e.g., prior to storage). In certain embodiments, the period of storage time is about 6 hours, about 12 hours, about 16 hours, about 24 hours, about 36 hours, about 48 hours, or greater than about 48 hours.

### A. Post-Compression Hardening Excipients

Excipients used to promote post-compression hardening post-compression can include two or more polyols co-processed to form a homogeneous material. Non-limiting examples of polyols include mannitol, sorbitol, maltitol, xylitol, erythritol, hydrogenated starch hydrolysates, isomalt, lactitol, and any derivative thereof. In certain embodiments, compositions of the present disclosure can also comprise one or more of sucrose, dextrose, maltose, microcrystalline cellulose, dicalcium phosphate anhydrous, dicalcium phosphate dihydrate, calcium phosphate, starch, pregelatinized starch, calcium carbonate, silicified microcrystalline cellulose, lactose anhydrous, lactose monohydrate, hydroxy propyl cellulose, and any derivative thereof.

In some embodiments, the compositions of the present disclosure comprise two polyols co-processed to form a homogeneous material (e.g., a first polyol and a second polyol). For example, a composition of the present disclosure can comprise sorbitol and maltitol. In another example, a composition of the present disclosure can comprise sorbitol and xylitol. In yet another example, a composition of the present disclosure can comprise sorbitol and erythritol. The ratio of the first polyol and the second polyol in the composition can be about 1000:1, about 500:1, about 250:1, about 100:1, about 50:1, about 20:1, about 10:1, about 5:1, about 4:1, about 3:1, about 2:1, about 1:1, about 1:2, about 1:3, about 1:4, about 1:5, about 1:10, about 1:20, about 1:50, about 1:100, about 1:250, about 1:500, or about 1:1000. In some embodiments, the compositions of the present disclosure comprise three polyols co-processed to form a homogeneous material. In some embodiments, the compositions of the present disclosure comprise more than three polyols co-processed to form a homogeneous material.

In certain embodiments, the composition can comprise a first polyol, and the first polyol can be present in the composition at about 0.1% by weight, about 0.5% by weight, about 1% by weight, about 5% by weight, about 10% by weight, about 15% by weight, about 20% by weight, about 25% by weight, about 30% by weight, about 35% by weight, about 40% by weight, about 45% by weight, or about 50% by weight. In certain embodiments, the composition can comprise a first polyol, and the first polyol can be present in the composition at about 0.1% by volume, about 0.5% by volume, about 1% by volume, about 5% by volume, about 10% by volume, about 15% by volume, about 20% by volume, about 25% by volume, about 30% by volume, about 35% by volume, about 40% by volume, about 45% by volume, or about 50% by volume.

In certain embodiments, the composition can comprise a second polyol, and the second polyol can be present in the composition at about 0.1% by weight, about 0.5% by weight, about 1% by weight, about 5% by weight, about 10% by weight, about 15% by weight, about 20% by weight, about 25% by weight, about 30% by weight, about 35% by weight, about 40% by weight, about 45% by weight, or about 50% by weight. In certain embodiments, the composition can comprise a second polyol, and the second polyol can be present in the composition at about 0.1% by volume, about 0.5% by volume, about 1% by volume, about 5% by volume, about 10% by volume, about 15% by volume, about 20% by volume, about 25% by volume, about 30% by volume, about 35% by volume, about 40% by volume, about 45% by volume, or about 50% by volume.

In one example, a composition of the present disclosure can comprise about 20 wt % of a first polyol that is sorbitol, and about 10 wt % of a second polyol that is maltitol. In another example, a composition of the present disclosure can comprise about 10 wt % of a first polyol that is sorbitol, and about 10 wt % of a second polyol that is maltitol. In yet another example, a composition of the present disclosure can comprise about 10 wt % of a first polyol that is sorbitol, and about 20 wt % of a second polyol that is maltitol. In some embodiments, compositions of the present disclosure can comprise 3 polyols. In one example, a composition of the present disclosure can comprise about 20 wt % of a first polyol that is sorbitol, about 10 wt % of a second polyol that is maltitol, and between about 60 wt % and about 70 wt % of a third polyol that is mannitol. In another example, a composition of the present disclosure can comprise about 10 wt % of a first polyol that is sorbitol, about 10 wt % of a second polyol that is maltitol, and between about 70 wt % and about 80 wt % of a third polyol that is mannitol. In yet another example, a composition of the present disclosure can comprise about 10 wt % of a first polyol that is sorbitol, about 20 wt % of a second polyol that is maltitol, and between about 60 wt % and 70 wt % of a third polyol that is mannitol. Table 29 provides a list of exemplary formulations comprising 3 polyols with various ratios of mannitol, sorbitol, and maltitol by weight.

TABLE 29
Formulations comprising Mannitol, Sorbitol, and Maltitol

| Formulation | (parts of 100) | | |
|---|---|---|---|
| # | Mannitol | Sorbitol | Maltitol |
| 001 | 65 | 25 | 10 |
| 002 | 65 | 24 | 11 |
| 003 | 65 | 23 | 12 |
| 004 | 65 | 22 | 13 |
| 005 | 65 | 21 | 14 |
| 006 | 65 | 20 | 15 |
| 007 | 65 | 19 | 16 |
| 008 | 65 | 18 | 17 |
| 009 | 65 | 17 | 18 |
| 010 | 65 | 16 | 19 |
| 011 | 65 | 15 | 20 |
| 012 | 65 | 14 | 21 |
| 013 | 65 | 13 | 22 |
| 014 | 65 | 12 | 23 |
| 015 | 65 | 11 | 24 |
| 016 | 65 | 10 | 25 |
| 017 | 66 | 25 | 11 |
| 018 | 66 | 24 | 12 |
| 019 | 66 | 23 | 13 |
| 020 | 66 | 22 | 14 |
| 021 | 66 | 21 | 15 |
| 022 | 66 | 20 | 16 |
| 023 | 66 | 19 | 17 |
| 024 | 66 | 18 | 18 |
| 025 | 66 | 17 | 19 |
| 026 | 66 | 16 | 20 |
| 027 | 66 | 15 | 21 |
| 028 | 66 | 14 | 22 |
| 029 | 66 | 13 | 23 |
| 030 | 66 | 12 | 24 |
| 031 | 66 | 11 | 25 |
| 032 | 67 | 25 | 8 |
| 033 | 67 | 24 | 9 |
| 034 | 67 | 23 | 10 |
| 035 | 67 | 22 | 11 |
| 036 | 67 | 21 | 12 |
| 037 | 67 | 20 | 13 |
| 038 | 67 | 19 | 14 |
| 039 | 67 | 18 | 15 |
| 040 | 67 | 17 | 16 |
| 041 | 67 | 16 | 17 |
| 042 | 67 | 15 | 18 |
| 043 | 67 | 14 | 19 |
| 044 | 67 | 13 | 20 |
| 045 | 67 | 12 | 21 |
| 046 | 67 | 11 | 22 |
| 047 | 67 | 10 | 23 |
| 048 | 67 | 9 | 24 |
| 049 | 67 | 8 | 25 |
| 050 | 68 | 22 | 10 |
| 051 | 68 | 21 | 11 |
| 052 | 68 | 20 | 12 |
| 053 | 68 | 19 | 13 |
| 054 | 68 | 18 | 14 |
| 055 | 68 | 17 | 15 |
| 056 | 68 | 16 | 16 |
| 057 | 68 | 15 | 17 |
| 058 | 68 | 14 | 18 |
| 059 | 68 | 13 | 19 |
| 060 | 68 | 12 | 20 |
| 061 | 68 | 11 | 21 |
| 062 | 68 | 10 | 22 |
| 063 | 69 | 21 | 10 |
| 064 | 69 | 20 | 11 |
| 065 | 69 | 19 | 12 |
| 066 | 69 | 18 | 13 |
| 067 | 69 | 17 | 14 |
| 068 | 69 | 16 | 15 |
| 069 | 69 | 15 | 16 |
| 070 | 69 | 14 | 17 |
| 071 | 69 | 13 | 18 |
| 072 | 69 | 12 | 19 |
| 073 | 69 | 11 | 20 |
| 074 | 69 | 10 | 21 |
| 075 | 70 | 20 | 10 |
| 076 | 70 | 19 | 11 |
| 077 | 70 | 18 | 12 |
| 078 | 70 | 17 | 13 |
| 079 | 70 | 16 | 14 |
| 080 | 70 | 15 | 15 |
| 081 | 70 | 14 | 16 |
| 082 | 70 | 13 | 17 |
| 083 | 70 | 12 | 18 |
| 084 | 70 | 11 | 19 |
| 085 | 70 | 10 | 20 |
| 086 | 71 | 19 | 10 |
| 087 | 71 | 18 | 11 |
| 088 | 71 | 17 | 12 |
| 089 | 71 | 16 | 13 |
| 090 | 71 | 15 | 14 |
| 091 | 71 | 14 | 15 |
| 092 | 71 | 13 | 16 |
| 093 | 71 | 12 | 17 |
| 094 | 71 | 11 | 18 |
| 095 | 71 | 10 | 19 |
| 096 | 72 | 20 | 8 |
| 097 | 72 | 19 | 9 |
| 098 | 72 | 18 | 10 |
| 99 | 72 | 17 | 11 |
| 100 | 72 | 16 | 12 |
| 101 | 72 | 15 | 13 |
| 102 | 72 | 14 | 14 |
| 103 | 72 | 13 | 15 |
| 104 | 72 | 12 | 16 |
| 105 | 72 | 11 | 17 |
| 106 | 72 | 10 | 18 |
| 107 | 72 | 9 | 19 |
| 108 | 72 | 8 | 20 |
| 109 | 73 | 19 | 8 |
| 110 | 73 | 18 | 9 |
| 111 | 73 | 17 | 10 |
| 112 | 73 | 16 | 11 |
| 113 | 73 | 15 | 12 |
| 114 | 73 | 14 | 13 |
| 115 | 73 | 13 | 14 |
| 116 | 73 | 12 | 15 |
| 117 | 73 | 11 | 16 |
| 118 | 73 | 10 | 17 |
| 119 | 73 | 9 | 18 |
| 120 | 73 | 8 | 19 |
| 121 | 74 | 18 | 8 |
| 122 | 74 | 17 | 9 |
| 123 | 74 | 16 | 10 |
| 124 | 74 | 15 | 11 |
| 125 | 74 | 14 | 12 |
| 126 | 74 | 13 | 13 |
| 127 | 74 | 12 | 14 |
| 128 | 74 | 11 | 15 |
| 129 | 74 | 10 | 16 |
| 130 | 74 | 9 | 17 |
| 131 | 74 | 8 | 18 |
| 132 | 75 | 17 | 8 |
| 133 | 75 | 16 | 9 |
| 134 | 75 | 15 | 10 |
| 135 | 75 | 14 | 11 |
| 136 | 75 | 13 | 12 |
| 137 | 75 | 12 | 13 |
| 138 | 75 | 11 | 14 |
| 139 | 75 | 10 | 15 |
| 140 | 75 | 9 | 16 |
| 141 | 75 | 8 | 17 |
| 142 | 76 | 17 | 7 |
| 143 | 76 | 16 | 8 |
| 144 | 76 | 15 | 9 |
| 145 | 76 | 14 | 10 |
| 146 | 76 | 13 | 11 |
| 147 | 76 | 12 | 12 |
| 148 | 76 | 11 | 13 |
| 149 | 76 | 10 | 14 |
| 150 | 76 | 9 | 15 |
| 151 | 76 | 8 | 16 |
| 152 | 76 | 7 | 17 |
| 153 | 77 | 16 | 7 |
| 154 | 77 | 15 | 8 |
| 155 | 77 | 14 | 9 |
| 156 | 77 | 13 | 10 |
| 157 | 77 | 12 | 11 |
| 158 | 77 | 11 | 12 |
| 159 | 77 | 10 | 13 |
| 160 | 77 | 9 | 14 |
| 161 | 77 | 8 | 15 |
| 162 | 77 | 7 | 16 |
| 163 | 78 | 16 | 6 |
| 164 | 78 | 15 | 7 |
| 165 | 78 | 14 | 8 |
| 166 | 78 | 13 | 9 |
| 167 | 78 | 12 | 10 |
| 168 | 78 | 11 | 11 |
| 169 | 78 | 10 | 12 |
| 170 | 78 | 9 | 13 |
| 171 | 78 | 8 | 14 |
| 172 | 78 | 7 | 15 |
| 173 | 78 | 6 | 16 |
| 174 | 79 | 15 | 6 |
| 175 | 79 | 14 | 7 |
| 176 | 79 | 13 | 8 |
| 177 | 79 | 12 | 9 |
| 178 | 79 | 11 | 10 |
| 179 | 79 | 10 | 11 |
| 180 | 79 | 9 | 12 |
| 181 | 79 | 8 | 13 |
| 182 | 79 | 7 | 14 |
| 183 | 79 | 6 | 15 |
| 184 | 80 | 15 | 5 |
| 185 | 80 | 14 | 6 |
| 186 | 80 | 13 | 7 |
| 187 | 80 | 12 | 8 |
| 188 | 80 | 11 | 9 |
| 189 | 80 | 10 | 10 |
| 190 | 80 | 9 | 11 |
| 191 | 80 | 8 | 12 |
| 192 | 80 | 7 | 13 |
| 193 | 80 | 6 | 14 |
| 194 | 80 | 5 | 15 |
| 195 | 81 | 14 | 6 |
| 196 | 81 | 13 | 7 |
| 197 | 81 | 12 | 8 |
| 198 | 81 | 11 | 9 |
| 199 | 81 | 10 | 10 |
| 200 | 81 | 9 | 11 |
| 201 | 81 | 8 | 12 |
| 202 | 81 | 7 | 13 |
| 203 | 81 | 6 | 14 |
| 204 | 82 | 13 | 5 |
| 205 | 82 | 12 | 6 |
| 206 | 82 | 11 | 7 |
| 207 | 82 | 10 | 8 |
| 208 | 82 | 9 | 9 |
| 209 | 82 | 8 | 10 |
| 210 | 82 | 7 | 11 |
| 211 | 82 | 6 | 12 |
| 212 | 82 | 5 | 13 |
| 213 | 83 | 13 | 4 |
| 214 | 83 | 12 | 5 |
| 215 | 83 | 11 | 6 |
| 216 | 83 | 10 | 7 |
| 217 | 83 | 9 | 8 |
| 218 | 83 | 8 | 9 |
| 219 | 83 | 7 | 10 |
| 220 | 83 | 6 | 11 |
| 221 | 83 | 5 | 12 |
| 222 | 83 | 4 | 13 |
| 223 | 84 | 12 | 4 |
| 224 | 84 | 11 | 5 |
| 225 | 84 | 10 | 6 |
| 226 | 84 | 9 | 7 |
| 227 | 84 | 8 | 8 |
| 228 | 84 | 7 | 9 |
| 229 | 84 | 6 | 10 |
| 230 | 84 | 5 | 11 |
| 231 | 84 | 4 | 12 |
| 232 | 85 | 11 | 4 |
| 233 | 85 | 10 | 5 |
| 234 | 85 | 9 | 6 |
| 235 | 85 | 8 | 7 |
| 236 | 85 | 7 | 8 |
| 237 | 85 | 6 | 9 |
| 238 | 85 | 5 | 10 |
| 239 | 85 | 4 | 11 |

In some embodiments, the present disclosure comprises an excipient system with significant post compression hardening, which extends to compression of difficult to compress active ingredients at high drug loadings. Soluble binders such as mannitol, lactose and sorbitol can have limited use as direct compression binders due to their relatively low tabletability. Embodiments of the present disclosure can overcome these drawbacks, making it possible to create robust tablets using a simple direct compression process with high levels of active ingredients contained therein, which have high levels of APIs. The resultant dosage forms have desirable hardness (>5 kP), Tensile Strength (>1.5 MPa) and Friability (<1%). Successful tablets have been produced across a range of active ingredients including acetaminophen, griseofulvin, acyclovir, and ibuprofen, with active ingredient levels of at least 20%, at least 30%, at least 40%, at least 50%, at least 60%, at least 70% or at least 80% by weight of the formulations. The resultant tablets are as robust as the equivalent tablets produced using a wet granulation process or using materials such as silicified microcrystalline cellulose or other forms of insoluble cellulose based binders. Tablets prepared using embodiments of the present disclosure as a soluble binder in combination with standard excipients that enable disintegration, and with high levels of API, have adequate disintegration and subsequent dissolution properties that are pre-requisites for the formulation of a dosage form that will meet monograph requirements. Such a soluble binder system may be used as a material that is soluble and can be compressed in a direct compression process without the need for other unit process steps, such as wet or dry granulation. In particular, the properties of the materials described herein are highly desirable for the manufacture of drugs that are high dose (e.g., greater than 100 mg, 200 mg, 300 mg, 400 mg, 500 mg, 800 mg or 1000 mg). Such drugs currently require granulation steps, which may be undesirable as the drugs may be moisture sensitive. Furthermore, embodiments of the present disclosure offer utility for drugs that chemically have primary or secondary amine groups which currently cannot be manufactured using alternative soluble binders (such as lactose) that lead to instability of drugs containing primary or secondary amines via the Maillard reaction.

When looking to formulate a drug at a high dose with a high drug loading, a granulation process (such as wet high shear or fluid bed or spray granulation techniques) are typically used. In these processes, a binding material such as polyvinyl pyrrolidone, HPMC or starch is sprayed onto the soluble binder such as lactose or mannitol and drug combination to increase the size of the individual components, but also to make the formed granules subsequently much more compactible. Using embodiments of the present disclosure, such processes are no longer required as the hardening phenomenon, as demonstrated herein, gives a soluble binder product that is highly compactible giving surprisingly robust tablets even at relatively low compaction forces.

In another embodiment, a tablet may be formulated utilizing a dry granulation process such as roller compaction or slugging by combining the drug with a material (binder) that renders it more compactible and able to form robust tablets. Materials used for such process includes insoluble binders such as microcrystalline cellulose. In yet another embodiment, direct compression processes are used by blending a drug that is poorly compactible or present in a high dose with an insoluble binder material such as microcrystalline cellulose (known as Avicel or Ceolus) or silicified microcrystalline cellulose (known as ProSolv).

There also exist some binder systems that are co-processed in an attempt to achieve the required compactibility of the drug and binding component. Examples of materials that exist include combinations of microcrystalline cellulose and mannitol (known as Avicel HFE), microcrystalline cellulose and lactose (known as Microcelac) and lactose povidone and copovidone (known as Ludipress). In certain embodiments, the co-spray dried material described herein surprisingly shows superior performance from a compactibility perspective to the range of materials described above, giving particularly high tablet robustness at low compression forces, whilst retaining certain disintegration and dissolution properties.

### B. Active Ingredients

A composition of the present disclosure can comprise one or more active ingredients. The compositions and methods of the present disclosure that uses both a high initial hardness upon compaction followed by a post-compaction hardening to increase tablet hardness are useful for preserving active ingredients that are pressure or moisture sensitive. By using hardening post-compression, instead of greater compression forces, lower compression forces can be used to manufacture various dosage forms at a lower water activity, thereby preserving the compression-sensitive or moisture-sensitive active ingredient.

### Pressure-Sensitive & Moisture-Sensitive Active Ingredients

A composition of the present disclosure can comprise a pressure-sensitive active ingredient. In certain embodiments, a pressure-sensitive active ingredient can comprise a probiotic. Non-limiting examples of a probiotic include Bacillus subtilis, Bacillus coagulans, Bifidobacterium adolescentis, Bifidobacterium animalis, Bifidobacterium bifidum, Bifidobacterium breve, Bifidobacterium infantis, Bifidobacterium longum, Bifidobacterium thermophilum, Lactobacillus acidophilus, Lactobacillus agilis, Lactobacillus alactosus, Lactobacillus alimentarius, Lactobacillus amylophilus, Lactobacillus amylovorans, Lactobacillus amylovorus, Lactobacillus animalis, Lactobacillus batatas, Lactobacillus bavaricus, Lactobacillus bifermentans, Lactobacillus bifidus, Lactobacillus brevis, Lactobacillus buchnerii, Lactobacillus bulgaricus, Lactobacillus catenaforme, Lactobacillus casei, Lactobacillus cellobiosus, Lactobacillus collinoides, Lactobacillus confusus, Lactobacillus coprophilus, Lactobacillus coryniformis, Lactobacillus corynoides, Lactobacillus crispatus, Lactobacillus curvatus, Lactobacillus delbrueckii, Lactobacillus desidiosus, Lactobacillus divergens, Lactobacillus enterii, Lactobacillus farciminis, Lactobacillus fermentum, Lactobacillus frigidus, Lactobacillus fructivorans, Lactobacillus fructosus, Lactobacillus gasseri, Lactobacillus halotolerans, Lactobacillus helveticus, Lactobacillus heterohiochii, Lactobacillus hilgardii, Lactobacillus hordniae, Lactobacillus inulinus, Lactobacillus jensenii, Lactobacillus jugurti, Lactobacillus kandleri, Lactobacillus kefir, Lactobacillus lactis, Lactobacillus leichmannii, Lactobacillus lindneri, Lactobacillus malefermentans, Lactobacillus mall, Lactobacillus maltaromicus, Lactobacillus minor, Lactobacillus minutus, Lactobacillus mobilis, Lactobacillus murinus, Lactobacillus pentosus, Lactobacillus plantarum, Lactobacillus pseudoplantarum, Lactobacillus reuteri, Lactobacillus rhamnosus, Lactobacillus rogosae, Lactobacillus tolerans, Lactobacillus torquens, Lactobacillus ruminis, Lactobacillus sake, Lactobacillus salivarius, Lactobacillus sanfrancisco, Lactobacillus sharpeae, Lactobacillus trichodes, Lactobacillus vaccinostercus, Lactobacillus viridescens, Lactobacillus vitulinus, Lactobacillus xylosus, Lactobacillus yamanashiensis, Lactobacillus zeae, Pediococcus acidilactici, Pediococcus pentosaceus, Streptococcus cremoris, Streptococcus diacetylactis, Streptococcus (Enterococcus) faecium, Streptococcus intermedius, Streptococcus lactis, Streptococcus thermophilus, and Saccharomyces boulardii. In certain embodiments, a composition of the present disclosure can comprise 2, 3, 4, 5, 6, 7, 8, 9, 10, or more than 10 probiotics in a single dosage form.

Numerous moisture-sensitive ingredients are known, such as amlodipine, angiotensin converting enzyme (ACE) inhibitor like Cilazapril, aspirin, atorvastatin, dabigatran, felodipine, fesoterodine fumarate, grizeofulvin, isradipine, itavastatin, lansoprazole, levothyroxine, lovastatin, niacinanimide, nifedipine, nimodipine, nisoldipine, omeprazole, pancreatine, pantoprazole, peptides, potassium clavulanate, pravastatin, proteins, rosuvastatin, simvastatin, tiotropium, and salts, esters, and solvates thereof. Moisture sensitivity is intended to encompass any undesired changes in an ingredient substance that occur as a result of exposure to moisture, such as atmospheric humidity. Such changes can involve ingredient compound degradation that forms one or more impurities, changes in physical characteristics, and/or morphological changes.

In some instances, stability of a moisture-sensitive ingredient is evidenced by a slow rate of degradant compound formation, over time. The period, during which an ingredient must remain stable, i.e., maintain its potency and/or impurity content in a formulation, varies according to commercial specifications set by the manufacturer. For example, a product might be required to maintain certain potency specifications for a period of six months, one year, two years, or some other time following manufacturing. The established shelf life of a product presumes maintenance in the original packaging, in specified temperature and humidity environments.

In certain embodiments, the pressure sensitive active could be a means of delivering certain lipophilic active substances in the form of a particle where the particle itself comprises of the lipid and API with or without a surfactant or surface active agent or emulsifier wherein that lipid system is in the form of a solid, semi-solid, or liquid when, in the form of a liquid the liquid may be adsorbed onto a carrier material such as microcrystalline cellulose, starch or silicon dioxide, magnesium aluminometasilicate to create the solid particle.

Other active ingredients can also be used in compositions of the present disclosure. Non-limiting examples of active ingredients suitable for use in the compositions of the present disclosure include cannabinoids from synthetic or from cannabis or hemp extracts, such as Cannabidiol (CBD), dronabinol, Cannabinol (CBN), Cannabichromene (CBC), Cannabigerol (CBG), Cannabidivarin (CBV), Hydrocodone/APAP (Brand Name: Vicodin®), Amoxicillin (Brand Name: Amoxil®), Lisinopril (Brand Name: Prinivil®), Esomeprazole (Brand Name: Nexium®), Atorvastatin (Brand Name: Lipitor®), Simvastatin (Brand Name: Zocor®), Clopidogrel (Brand Name: Plavix®), Montelukast (Brand Name: Singulair®), Rosuvastatin (Brand Name: Crestor®), Metoprolol (Brand Name: Lopressor®), Escitalopram (Brand Name: Lexapro®), Azithromycin (Brand Name: Zithromax®), Albuterol (Brand Name: ProAir® HFA), Hydrochlorothiazide (Brand Name: HCTZ), Metformin (Brand Name: Glucophage®), Sertraline (Brand Name: Zoloft®), Ibuprofen (Brand Name: Advil®), Zolpidem (Brand Name: Ambien®), Furosemide (Brand Name: Lasix®), Omeprazole (Brand Name: Prilosec®), Trazodone (Brand Name: Desyrel®), Valsartan (Brand Name: Diovan®), Tramadol (Ultram®), Duloxetine (Brand Name: Cymbalta®), Warfarin (Brand Name: Coumadin®), Amlodipine (Brand Name: Norvasc®), Oxycodone/APAP (Brand Name: Percocet®), Quetiapine (Brand Name: Seroquel®), Promethazine (Brand Name: Phenergan®), Fluticasone (Brand Name: Flonase®), Alprazolam (Brand Name: Xanax®), Clonazepam (Brand Name: Klonopin®), Benazepril (Brand Name: Lotensin®), Meloxicam (Brand Name: Mobic®), Citalopram (Brand Name: Celexa®), Cephalexin (Brand Name: Keflex®), Tiotropium (Brand Name: Spiriva®), Gabapentin (Brand Name: Neurontin®), Aripiprazole (Brand Name: Abilify®), Cyclobenzaprine (Brand Name: Flexeril®), Methylprednisolone (Brand Name: Medrol®), Methylphenidate (Brand Name: Ritalin®), Fexofenadine (Brand Name: Allegra®), Carvedilol (Brand Name: Coreg®), Carisoprodol (Brand Name: Soma®), Digoxin (Brand Name: Lanoxin®), Memantine (Brand Name: Namenda®), Atenolol (Brand Name: Tenormin®), Diazepam (Brand Name: Valium®), Oxycodone (Brand Name: OxyContin®), Risedronate (Brand Name: Actonel®), Folic Acid (Brand Name: Folvite®), Olmesartan (Brand Name: Benicar®), Prednisone (Brand Name: Deltasone®), Doxycycline (Brand Name: Vibramycin®), Alendronate (Brand Name: Fosamax®), Pantoprazole (Brand Name: Protonix®), Tamsulosin (Brand Name: Flomax®), Triamterene/HCTZ (Brand Name: Dyazide®), Paroxetine (Brand Name: Paxil®), Buprenorphine (Brand Name: Suboxone®), Enalapril (Brand Name: Vasotec®), Lovastatin (Brand Name: Mevacor®), Pioglitazone (Brand Name: Actos®), Pravastatin (Brand Name: Pravachol®), Fluoxetine (Brand Name: Prozac®), Insulin Detemir (Brand Name: Levemir®), Fluconazole (Brand Name: Diflucan®), Levofloxacin (Brand Name: Levaquin®), Rivaroxaban (Brand Name: Xarelto®), Celecoxib (Brand Name: Celebrex®), Codeine/APAP (Brand Name: Tylenol® #2), Mometasone (Brand Name: Nasonex®), Ciprofloxacin (Brand Name: Cipro®), Insulin Aspart (Novolog®), Venlafaxine (Brand Name: Effexor®), Lorazepam (Brand Name: Ativan®), Ezetimibe (Brand Name: Zetia®), Estrogen (Brand Name: Premarin®), Allopurinol (Brand Name: Zyloprim®), Penicillin (Brand Name: Pen VK®), Sitagliptin (Brand Name: Januvia®), Amitriptyline (Brand Name: Elavil®), Clonidine (Brand Name: Catapres®), Latanoprost (Brand Name: Xalatan®), Lisdexamfetamine (Brand Name: Vyvanse®), Niacin (Brand Name: Niaspan®), Naproxen (Brand Name: Aleve®), Dexlansoprazole (Brand Name: Dexilant®), Glyburide (Brand Name: Diabeta®), Olanzapine (Brand Name: Zyprexa®), Tolterodine (Brand Name: Detrol®), Ranitidine (Brand Name: Zantac®), Famotidine (Brand Name: Pepcid®), Diltiazem (Brand Name: Cardizem®), Insulin Glargine (Brand Name: Lantus®), Thyroid (Brand Name: Armour Thyroid®), Bupropion (Brand Name: Wellbutrin®), Cetirizine (Zyrtec®), Topiramate (Brand Name: Topamax®), Valacyclovir (Brand Name: Valtrex®), Eszopiclone (Brand Name: Lunesta®), Acyclovir (Brand Name: Zovirax®), Cefdinir (Brand Name: Omnicef®), Clindamycin (Brand Name: Cleocin®), Colchicine (Brand Name: Colcrys®), Gemfibrozil (Brand Name: Lopid®), Guaifenesin (Brand Name: Robitussin®), Glipizide (Brand Name: Glucotrol®), Irbesartan (Brand Name: Avapro®), Metoclopramide (Brand Name: Reglan®), Losartan (Brand Name: Cozaar®), Meclizine (Brand Name: Dramamine®), Metronidazole (Brand Name: Flagyl®), Vitamin D (Brand Name: Caltrate®), Testosterone (Brand Name: AndroGel®), Ropinirole (Brand Name: Requip®), Olopatadine (Brand Name: Patanol®), Moxifloxacin (Brand Name: Avelox®), Enoxaparin (Brand Name: Lovenox®), Fentanyl (Brand Name: Duragesic®), Dicyclomine (Brand Name: Bentyl®), Bisoprolol (Brand Name: Zebeta®), Atomoxetine (Brand Name: Strattera®), Ramipril (Brand Name: Altace®), Temazepam (Brand Name: Restoril®), Phentermine (Brand Name: Adipex® P), Quinapril (Brand Name: Accupril®), Sildenafil (Brand Name: Viagra®), Ondansetron (Brand Name: Zofiran®), Oseltamivir (Brand Name: Tamiflu®), Methotrexate (Brand Name: Rheumatrex®), Dabigatran (Brand Name: Pradaxa®), Budesonide (Brand Name: Uceris®), Doxazosin (Brand Name: Cardura®), Desvenlafaxine (Brand Name: Pristiq®), Insulin Lispro (Brand Name: Humalog®), Clarithromycin (Brand Name: Biaxin®), Buspirone (Brand Name: Buspar®), Finasteride (Brand Name: Proscar®), Ketoconazole (Brand Name: Nizoral®), Solifenacin (Brand Name: VESIcare®), Methadone (Brand Name: Dolophine®), Minocycline (Brand Name: Minocin®), Phenazopyridine (Brand Name: Pyridium®), Spironolactone (Brand Name: Aldactone®), Vardenafil (Brand Name: Levitra®), Clobetasol (Brand Name: Clovate®), Benzonatate (Brand Name: Tessalon®), Divalproex (Brand Name: Depakote®), Dutasteride (Brand Name: Avodart®), Febuxostat (Brand Name: Uloric®), Lamotrigine (Brand Name: Lamictal®), Nortriptyline (Brand Name: Pamelor®), Roflumilast (Brand Name: Daliresp®), Rabeprazole (Brand Name: Aciphex®), Etanercept (Brand Name: Enbrel®), Nebivolol (Brand Name: Bystolic®), Nabumetone (Brand Name: Relafen®), Nifedipine (Brand Name: Procardia®), Nitrofurantoin (Brand Name: Macrobid®), Nitroglycerine (Brand Name: NitroStat® SL), Oxybutynin (Brand Name: Ditropan®), Tadalifil (Brand Name: Cialis®), Triamcinolone (Brand Name: Kenalog®), Rivastigmine (Brand Name: Exelon®), Lansoprazole (Brand Name: Prevacid®), Cefuroxime (Brand Name: Ceftin®), Methocarbamol (Brand Name: Robaxin®), Travoprost (Brand Name: Travatan®), Lurasidone (Brand Name: Latuda®), Terazosin (Brand Name: Hytrin®), Sumatriptan (Brand Name: Imitrex®), Raloxifene (Brand Name: Evista®), Mirtazepine (Brand Name: Remeron®), Adalimumab (Brand Name: Humira®), Benztropine (Brand Name: Cogentin®), Baclofen (Brand Name: Gablofen®), Hydralazine (Brand Name: Apresoline®), Mupirocin (Brand Name: Bactroban®), Propranolol (Brand Name: Inderal®), Varenicline (Brand Name: Chantix®), Verapamil (Brand Name: Verelan®), Clotrimazole (Brand Name: Lotrimin®), Phenytoin (Brand Name: Dilantin®), Pramipexole (Brand Name: Mirapex®), Liraglutide (Brand Name: Victoza®), Ticagrelor (Brand Name: Brilinta®), Diclofenac (Brand Name: Voltaren®), Saxagliptin (Brand Name: Onglyza®), Lomitapide (Brand Name: Juxtapid®), Tizanidine (Brand Name: Zanaflex®), Amphetamine/Dextroamphetamine (Brand Name: Adderall®), Zoster Vaccine (Brand Name: Zostavax®), Ezetimibe/Simvastatin (Brand Name: Vytorin®), Vilazodone (Brand Name: Vybriid®), Hydroxyzine (Brand Name: Vistaril®), Donepezil (Brand Name: Aricept®), Acetaminophen (Brand Name: Tylenol®), Oxcarbazepine (Brand Name: Trileptal®), and derivatives of any of the above, and combinations of any of the above.

### C. Other Additives

It is contemplated that, compositions of the present disclosure can comprise other additives (e.g., for preserving or cushioning an active ingredient, or for flavoring). Additives and inactive ingredients can include, but are not limited to binding materials, dyes, preservatives, and flavoring agents. Non-limiting examples of additives or inactive ingredients include acacia, acesulfame, acesulfame potassium, acetic acid, acetone, acetyltributyl citrate, alcohol, alginic acid, alpha-tocopherol, aluminum chloride, aluminum chlorohydrex propylene glycol, aluminum hydroxide, aluminum lake dyes, aluminum oxide, aluminum silicate, aluminum stearate, aluminum sulfate, amide resin, aminobenzoate sodium, ammonia, ammonio methacrylate copolymer, ammonio methacrylate copolymer type A, ammonio methacrylate copolymer type B, ammonio methacrylate copolymers, ammonium chloride, ammonium hydroxide, ammonium laureth-5 sulfate, ammonium phosphate dibasic, artificial flavor, artificial grape flavor, artificial mint flavor, ascorbic acid, ascorbyl palmitate, aspartame, aspartame powder, banana, barium sulfate, benzalkonium chloride, benzoic acid, benzyl alcohol, betadex, black currant, black currant flavor, black ink, black pigment, blackberry, blue dye, butyl alcohol, butylated hydroxyanisole, butylated hydroxytoluene, butylparaben, calcium, calcium carbonate, calcium phosphate, calcium phosphate dibasic anhydrous, calcium phosphate dihydrate dibasic, calcium silicate, calcium stearate, calcium sulfate, calcium sulfate anhydrous, calcium sulfate dihydrate, candelilla wax, candelilla wax powder, carbomer, carbomer 934, carbomer 934p, carbomer homopolymer type A, carbomer homopolymer type B, carbomer homopolymer type C, carboxymethylcellulose, carboxymethylcellulose calcium, carboxymethylcellulose sodium, carmine, carnauba wax, carrageenan, castor oil, castor wax, cellacefate, cellulose, cellulose acetate, cellulose compounds, cellulose powdered, cellulosic polymers, cetostearyl alcohol, cetyl alcohol, cetylpyridinium chloride, cherry, citric acid, citric acid anhydrous, citric acid monohydrate, cochineal, coconut oil, colophony, colorants, coloring agent, compressible sucrose, compressible sugar, confectioners sugar, copovidone, corn, corn oil, corn starch, corn syrup, corn syrup solids, corn-derived proteins, cottonseed oil, cranberry, croscarmellose sodium, croscarmellose sodium type A, crospovidone, cysteine hydrochloride, D&C Blue No. 1, D&C Green No. 5, D&C Red No. 21, D&C Red No. 22, D&C Red No. 27, D&C Red No. 27 Aluminum Lake, D&C Red No. 27 Lake, D&C Red No. 28, D&C Red No. 28 Aluminum Lake, D&C Red No. 30, D&C Red No. 30 Aluminum Lake, D&C Red No. 33, D&C Red No. 40, D&C Red No. 6, D&C Red No. 6 Lake, D&C Red No. 7, D&C Red No. 7 Calcium Lake, D&C Yellow No. 10, D&C Yellow No. 10 Aluminum Lake, D&C Yellow No. 10 Lake, D&C Yellow No. 5, D&C Yellow No. 6, dehydrated alcohol, dextrates, dextrose, dextrose monohydrate, dibasic calcium phosphate, dibutyl phthalate, dibutyl sebacate, dicalcium phosphate, diethyl phthalate, dihydroxyaluminum sodium carbonate, dimethicone, dimethylaminoethyl methacrylate-butyl methacrylate-methyl methacrylate copolymer, dimethylpolysiloxane, docusate sodium, dyes, edetate calcium disodium, edetate disodium, edible black ink, egg lecithin, erythrosine, erythrosine sodium, ethanolamine, ethyl acrylate-methyl methacrylate copolymer, ethyl alcohol, ethyl butyrate, ethyl isovalerate, ethylcellulose, ethylcellulose (10 mPa·s), ethylcellulose (100 mPa·s), ethylcellulose (20 mPa·s), ethylcellulose (7 mPa·s), ethylcelluloses, ethylene glycol monoethyl ether, ethylvanillin, eudragit, FD&C Blue No. 1, FD&C Blue No. 1 Aluminium Lake, FD&C Blue No. 1 Lake, FD&C Blue No. 2, FD&C Blue No. 2 Aluminium Lake, FD&C Blue No. 2 Lake, FD&C Green No. 3, FD&C Green No. 3 Aluminum Lake, FD&C Red No. 3, FD&C Red No. 4, FD&C Red No. 40, FD&C Red No. 40 Aluminium Lake, FD&C Red No. 40 Lake, FD&C Yellow No. 10, FD&C Yellow No. 10 Aluminum Lake, FD&C Yellow No. 10 Lake, FD&C Yellow No. 5, FD&C Yellow No. 5 Aluminum Lake, FD&C Yellow No. 5 Lake, FD&C Yellow No. 6, FD&C Yellow No. 6 Aluminum Lake, FD&C Yellow No. 6 Lake, ferric oxide, ferric oxide black, ferric oxide brown, ferric oxide orange, ferric oxide red, ferric oxide yellow, ferric oxides, ferrosoferric oxide, ferrous fumarate, ferrous oxide, flavor, flavors, fragrances, fumaric acid, fumed silica, gelatin, glucosamine, glucosamine hydrochloride, glutamic acid hydrochloride, glycerin, glycerol, glycerol monooleate, glycerol monostearate, glyceryl behenate, glyceryl distearate, glyceryl monooleate, glyceryl monostearate, glyceryl triacetate, glycine, glycolate, glycyrrhizin ammoniated, guar gum, hard gelatin capsule, hard paraffin, hydrochloric acid, hydrocloric acid, hydrogen peroxide, hydrogenated castor oil, hydrogenated cottonseed oil, hydrogenated soy oil, hydrogenated soybean oil, hydrogenated vegetable oil, hydroxyethyl cellulose, hydroxypropyl cellulose, hydroxypropyl methylcellulose, hydroxypropyl methylcellulose phthalate, hypromellose, hypromellose 2208, hypromellose 2208 (100 mPa·s), hypromellose 2208 (100000 mPa·s), hypromellose 2208 (15000 mPa·s), hypromellose 2208 (3 mPa·s), hypromellose 2208 (4000 mPa·s), hypromellose 2910, hypromellose 2910 (15 mPa·s), hypromellose 2910 (15000 mPa·s), hypromellose 2910 (3 mPa·s), hypromellose 2910 (5 mPa·s), hypromellose 2910 (50 mPa·s), hypromellose 2910 (6 mPa·s), hypromellose 2910 3 cp, hypromellose 2910 50 cp, hypromellose 2910 5 cp, hypromellose 2910 6 cp, hypromellose 3 cp, hypromellose 5 cp, hypromellose 6 cp, hypromellose phthalate, hypromelloses, indigotindisulfonate sodium, iron, isobutylparaben, isopropyl, isopropyl alcohol, lactitol, lactitol monohydrate, lactose, lactose anhydrous, lactose hydrous, lactose monohydrate, lecithin, lemon oil, leucine, light mineral oil, low substituted hydroxypropyl cellulose, magnesium, magnesium aluminum silicate, magnesium carbonate, magnesium hydroxide, magnesium oxide, magnesium oxide heavy, magnesium silicate, magnesium stearate, magnesium trisilicate, maleic acid, malic acid, maltodextrin, mannitol, medium-chain triglycerides, meglumine, menthol, mesoporous silica gel, methacrylic acid, methacrylic acid-ethyl acrylate copolymer (1:1) type a, methacrylic acid-methyl methacrylate copolymer (1:1), methacrylic acid-methyl methacrylate copolymer (1:2), methacrylic acid copolymer, methacrylic acid copolymer type B, methanol, methyl alcohol, methyl cinnamate, methyl methacrylate, methylcellulose, methylcellulose (100 mPa·s), methylcellulose (15 mPa·s), methylcellulose (400 mPa·s), methylene chloride, methylparaben, methylparaben sodium, microcrystalline cellulose, microcrystalline wax, mineral oil, mint, mint cream flavor, mint menthol, modified corn starch, monosodium citrate, natural and artificial orange flavor, natural flavor, natural mint flavor, natural peppermint flavor, natural resin, nonoxynol-100, oleic acid, olive oil, opacode black, orange cream flavor, orange juice, orange oil, orange-pineapple flavor, other ingredients, palm kernel oil, paraffin, partially hydrogenated soybean and palm oils, peanut oil, peppermint, peppermint flavor, peppermint oil, pharmaceutical glaze, phenylalanine, phosphoric acid, piperazine, polacrilin potassium, polacrilin sodium, poloxamer, poloxamer 188, poloxamer 407, polyacrylate dispersion 30%, polycarbophil, polydextrose, polyethylene glycol, polyethylene glycol 1450, polyethylene glycol 300, polyethylene glycol 3000, polyethylene glycol 3350, polyethylene glycol 400, polyethylene glycol 4000, polyethylene glycol 600, polyethylene glycol 6000, polyethylene glycol 800, polyethylene glycol 8000, polygalacturonic acid, polyplasdone xl, polysorbate, polysorbate 20, polysorbate 80, polyvinyl acetate, polyvinyl alcohol, polyvinylpyrrolidone, potassium, potassium bicarbonate, potassium bitartrate, potassium carbonate, potassium carbonate anhydrous, potassium chloride, potassium gluconate, potassium hydroxide, potassium sorbate, potato starch, povidone, povidone k12, povidone k25, povidone k29/32, povidone k30, povidone k90, precipitated calcium carbonate, pregelatinized corn starch, pregelatinized starch, propyl gallate, propylene glycol, propylene glycol alginate, propylparaben, propylparaben sodium, raspberry, raw sugar, riboflavin, rice starch, saccharin, saccharin sodium, sd-45 alcohol, sda-3a alcohol, sesame oil, shellac, silicified microcrystalline cellulose, silicon dioxide, silicon dioxide colloidal, silicone, simethicone, simethicone emulsion, sodium, sodium alginate, sodium ascorbate, sodium benzoate, sodium bicarbonate, sodium carbonate, sodium carbonate monohydrate, sodium caseinate, sodium chloride, sodium citrate, sodium citrate dihydrate, sodium glycolate, sodium hydroxide, sodium laureth sulfate, sodium lauryl sulfate, sodium lauryl sulphate, sodium metabisulfite, sodium monolaurate, sodium phosphate, sodium phosphate dibasic, sodium propionate, sodium starch glycolate, sodium starch glycolate type A potato, sodium stearate, sodium stearyl fumarate, sodium thioglycolate, sodium tripolyphosphate, sorbic acid, sorbitan, sorbitan monolaurate, sorbitan monooleate, sorbitol, sorbitol special, soya lecithin, soybean oil, spearmint, starch, stearic acid, stearyl alcohol, strawberry, strawberry guarana flavor, strong ammonia solution, succinic acid, sucralose, sucrose, sucrose stearate, sugar 6× powder, sugar spheres, sunflower oil, synthetic ferric oxide, synthetic ferric oxide black, synthetic ferric oxide red, synthetic ferric oxide yellow, synthetic ferric oxides, tapioca starch, tartaric acid, tartrazine, taurine, TIMERx-N, titanium dioxide, titanium oxide, tragacanth, triacetin, tribehenin, tricalcium phosphate, triethyl citrate, trimyristin, trisodium citrate anhydrous, trisodium citrate dihydrate, tromethamine, tropical blend flavor, vanilla, vanilla flavor, vanillin, vitamin e, water, wax, wheat starch, white wax, xanthan gum, xylitol, yellow wax, zinc gluconate, and zinc stearate.

In some cases, the compositions described herein may comprise an additional excipient (e.g., separate from the post-compression hardening excipients described above) that can provide long term preservation, bulk up a formulation that contains potent active ingredients, facilitate drug absorption, reduce viscosity, add flavoring, or enhance the solubility of the composition. Non-limiting examples of excipients can include anti-adherents, binders (e.g., sucrose, lactose, starches, cellulose, gelatin, or polyethylene glycol), coatings (e.g., hydroxypropyl methylcellulose or gelatin), disintegrants, dyes, flavors (e.g., mint, peach, raspberry, or vanilla), glidants, lubricants, preservatives (e.g., acids, esters, phenols, mercurial compounds, or ammonium compounds), sorbents, or drug delivery vehicles (e.g., petroleum or mineral oil). A composition of the present disclosure can comprise about 1%, 2%, 3%, 4%, 5%, 6%, 7%, 8%, 9%, 10%1, 15%, 20%, 25%, 30%, 35%, 40%, 45%, 50%, or greater than about 50% of the excipient by weight or by volume. For example, a composition can comprise 5% of an excipient by volume.

In certain embodiments, compositions of the present disclosure can comprise one or more lubricants. Non-limiting examples of lubricants include Boric acid, Magnesium stearate, Calcium stearate, sodium stearyl fumarate, Sodium stearate, Carbowax (PEG) 4000-6000, Stearic acid, Sodium oleate, Sterotex, Sodium benzoate, Talc, Sodium acetate, Waxes, Sodium lauryl sulfate, Stear-O-Wet, Magnesium Lauryl sulfate, Glyceryl behenate, and Hydrogenated oil. The concentration of the lubricant can be between about 0.1% and 5% of the total composition by weight, wherein the total composition comprises two or more polyols (e.g., mannitol, sorbitol, and maltitol in a ratio of 85:10:5 by weight), the salt or the lubricant (e.g., magnesium stearate), optionally one or more selected from silica gel, fumed silica, colloidal silica, magnesium aluminometasilicate and silicon dioxide (e.g., Syloid 3150), optionally an active ingredient, and optionally an excipient. For example, a composition of the present disclosure can comprise about 83% by weight of mannitol, sorbitol, and maltitol in a ratio of 85:10:5 by weight, about 2% magnesium stearate, and about 15% by weight of Syloid 3150. Exemplary embodiments of compositions of the present disclosure can comprise about 1% by weight of Boric acid, about 0.25% to about 2% by weight of magnesium stearate, about 0.25% to about 2% by weight of calcium stearate, about 0.25% to about 2% by weight of sodium stearate, about 0.25-2.5% sodium stearyl fumarate, about 1% to about 5% by weight of Carbowax (PEG) 4000-6000, about 0.25% to about 2% by weight of Stearic acid, about 5% by weight of Sodium oleate, about 0.25% to about 1% by weight of Sterotex, about 5% by weight of Sodium benzoate, about 1% to about 5% by weight of Talc, about 5% by weight of Sodium acetate, about 1% to about 5% by weight of Wax, about 1% to about 5% by weight of Sodium lauryl sulfate, about 1% to about 5% by weight of Stear-O-Wet, about 1% to about 2% by weight of Magnesium Lauryl sulfate, about 0.5% to about 3% by weight of Glyceryl behenate, and/or about 1% to about 5% by weight of Hydrogenated oil.

In another example, a composition can comprise 10% of an excipient by weight. It is contemplated that one or more delivery vehicles can be chosen based on the active ingredient in the composition. Accordingly, a delivery vehicle can be chosen, for example, to improve efficacy of an active ingredient, prevent degradation and/or increase half-life of an active ingredient, reduce toxicity, and/or reduce immunogenicity. It is also contemplated that one or more delivery vehicles can be chosen to control the concentration of the active ingredient (e.g., a delivery vehicle capable of delivering a higher dose of an active ingredient in a single administration of the composition). Exemplary vehicles can include, but are not limited, one or more polymers (e.g., polyethylene glycol (PEG)), polylysine, dextran, lipids, cholesterol groups, steroids, carbohydrates, and oligosaccharides.

In certain embodiments, a composition of the present disclosure can comprise one or more solubilizers. As used herein, “solubilizers” include compounds such as triacetin, triethylcitrate, ethyl oleate, ethyl caprylate, sodium lauryl sulfate, sodium docusate, vitamin E TPGS, dimethylacetamide, N-methylpyrrohdone, N-hydroxyethylpyrrolidone, polyvinylpyrrolidone, hydroxypropylmethyl cellulose, hydroxypropyl cyclodextrins, ethanol, n-butanol isopropyl alcohol, cholesterol, bile salts, polyethylene glycol 200-600, glycofurol, propylene glycol, and dimethyl isosorbide and the like. A composition of the present disclosure can comprise about 1%, 2%, 3%, 4%, 5%, 6%, 7%, 8%, 9%, 10%, 15%, 20%, 25%, 30%, 35%, 40%, 45%, 50%, or greater than about 50% of the solubilizer by weight or by volume. For example, a composition can comprise 10% of a solubilizer by volume. In another example, a composition can comprise 5% of a solubilizer by weight.

In some embodiments, the compositions described herein include excipients, other medicinal or pharmaceutical agents, carriers, adjuvants, such as preserving, stabilizing, wetting or emulsifying agents, solution promoters, and salts for regulating the osmotic pressure, osmolarity, and/or osmolality of the composition. In other embodiments, the excipients, carriers, adjuvants, are useful in forming a pharmaceutically acceptable thickened composition. In some embodiments, the compositions comprise a stabilizing agent. In some embodiments, stabilizing agent is selected from, for example, fatty acids, fatty alcohols, alcohols, long chain fatty acid esters, long chain ethers, hydrophilic derivatives of fatty acids, polyvinyl pyrrolidones, polyvinyl ethers, polyvinyl alcohols, hydrocarbons, hydrophobic polymers, moisture-absorbing polymers, and combinations thereof. In some embodiments, amide analogues of stabilizers are also used. In a further embodiment, the chosen stabilizer changes the hydrophobicity of the composition (e.g., oleic acid, waxes), or improves the mixing of various components in the composition (e.g., ethanol), controls the moisture level in the formula (e.g., PVP or polyvinyl pyrrolidone), controls the mobility of the phase (substances with melting points higher than room temperature such as long chain fatty acids, alcohols, esters, ethers, amides etc. or mixtures thereof; waxes), and/or improves the compatibility of the formula with a fluid delivery device of the present disclosure. In another embodiment, some of these stabilizers are used as solvents/co-solvents (e.g., ethanol). Other useful compositions include one or more antioxidants to enhance chemical stability where required. Suitable antioxidants include, by way of example only, ascorbic acid and sodium metabisulfite. In one embodiment, antioxidants are selected from metal chelating agents, thiol containing compounds and other general stabilizing agents. In one embodiment, using mesoporous silica gel, fumed silica as desiccants or API stabilizers or carriers.

Still other useful compositions include one or more surfactants to enhance physical stability or for other purposes. Suitable nonionic surfactants include polyoxyethylene fatty acid glycerides and vegetable oils, polyoxyethylene, hydrogenated castor oil, polyoxyethylene alkylethers, alkylphenyl ethers, octoxynol 10, and octoxynol 40.

In some embodiments, the composition comprises tablet binders, granule binders and surfactants. Useful tableting and granulation binders include for example, compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400. Other binders include sodium carboxymethylcellulose, methylcellulose, hydroxypropyl methylcellulose, hydroxy methylcellulose acetate stearate, hydroxyethyl cellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropyl methylcellulose, hydroxyethyl cellulose, sodium alginate, polyethoxylated sorbitan monolaurate, polyethoxylated sorbitan monolaurate, povidone and the like. In some embodiments, useful aqueous binders also contain one or more polymers as suspending agents. Useful polymers include water-soluble polymers such as cellulosic polymers, e.g., hydroxypropyl methylcellulose, and water-insoluble polymers such as cross-linked carboxyl-containing polymers.

In some embodiments, the composition comprises an additional surfactant (co-surfactant) and/or buffering agent and/or solvent. In some embodiments, the surfactant and/or buffering agent and/or solvent is a) natural and synthetic lipophilic agents, e.g., phospholipids, cholesterol, and cholesterol fatty acid esters and derivatives thereof; b) nonionic surfactants, which include for example, polyoxyethylene fatty alcohol esters, sorbitan fatty acid esters (Spans), polyoxyethylene sorbitan fatty acid esters (e.g., polyoxyethylene (20) sorbitan monooleate (Tween 80), polyoxyethylene (20) sorbitan monostearate (Tween 60), polyoxyethylene (20) sorbitan monolaurate (Tween 20) and other Tweens, sorbitan esters, glycerol esters, e.g., Myrj and glycerol triacetate (triacetin), polyethylene glycols, cetyl alcohol, cetostearyl alcohol, stearyl alcohol, polysorbate 80, poloxamers, poloxamines, polyoxyethylene castor oil derivatives (e.g., Cremophor® RH40, Cremphor A25, Cremphor A20, Cremophor® EL) and other Cremophors, sulfosuccinates, alkyl sulphates (SLS); PEG glyceryl fatty acid esters such as PEG-8 glyceryl caprylate/caprate (Labrasol), PEG-4 glyceryl caprylate/caprate (Labrafac Hydro WL 1219), PEG-32 glyceryl laurate (Gelucire 444/14), PEG-6 glyceryl mono oleate (Labrafil M 1944 CS), PEG-6 glyceryl linoleate (Labrafil M 2125 CS); propylene glycol mono- and di-fatty acid esters, such as propylene glycol laurate, propylene glycol caprylate/caprate; Brij® 700, ascorbyl-6-palmitate, stearylamine, sodium lauryl sulfate, polyoxethyleneglycerol triiricinoleate, and any combinations or mixtures thereof; c) anionic surfactants include, but are not limited to, calcium carboxymethylcellulose, sodium carboxymethylcelhilose, sodium sulfosuccinate, dioctyl, sodium alginate, alkyl polyoxyethylene sulfates, sodium lauryl sulfate, triethanolamine stearate, potassium laurate, bile salts, and any combinations or mixtures thereof; and d) cationic surfactants such as quatemary ammonium compounds, benzalkonium chloride, cetyltrimethylammonium bromide, and lauryldimethylbenzyl-ammonium chloride.

In some embodiments, the compositions described herein comprise a diluent. In some embodiments, the diluent is a salt (e.g., sodium chloride) dissolved in solution (e.g. phosphate buffered saline solution), lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropyhnethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, or combinations thereof. A composition of the present disclosure can comprise about 1%, 2%, 3%, 4%, 5%, 6%, 7%, 8%, 9% 10%, 15%, 20%, 25%, 30%, 35%, 40%, 45%, 50%, or greater than about 50% of the diluent by weight or by volume. For example, a composition can comprise 5% of a diluent by volume. In another example, a composition can comprise 8% of a diluent by weight.

In certain embodiments, the compositions of the present disclosure can comprise a plurality of vehicles, excipients, carriers, solubilizers, and the like. In any embodiment, the ratio (volume by volume or weight by weight) of a first vehicle, excipient, carrier, or solubilizer to a second vehicle, excipient, carrier, or solubilizer is less than about 1:10000, about 1:10000, about 1:5000, about 1:2500, about 1:1000, about 1:500, about 1:250, about 1:200, about 1:150, about 1:100, about 1:90, about 1:80, about 1:70, about 1:60, about 1:50, about 1:40, about 1:30, about 25:1, about 1:20, about 1:10, about 1:5, about 1:1, about 5:1, about 10:1, about 15:1, about 20:1, about 25:1, about 30:1, about 40:1, about 50:1, about 60:1, about 70:1, about 80:1, about 90:1, about 100:1, about 150:1, about 200:1, about 250:1, about 500:1, about 1000:1, about 2500:1, about 5000:1, about 10000:1, or greater than about 10000:1.

### Methods

The present disclosure provides methods for utilizing hardening to increase hardness of a dosage form comprising two or more excipients co-processed to form a homogeneous material (e.g., two or more polyols) post-compression (e.g., post-compaction). In particular, the methods of the present disclosure (e.g., for post-compression hardening of a dosage form) can be performed in the absence of moisture and/or heat. In certain embodiments, the co-processing can be selected from the group consisting of spray drying, spray congealing, granulation, lyophilization, fluid bed granulation, extrusion spherization, and chilsonation.

In one embodiment using the spray drying process, the powder is manufactured in a Buchi Mini Spray Dryer B-290. A 40% solids solution was made in a 400-600 ml beaker atop a Thermo Fisher Cimarec+SP88857100 Hotplate/Stir plate. The different ratio mixtures of Mannitol/Sorbitol/Maltitol were added to 180 g H₂O while agitating with a stir bar and heating in a covered beaker to approx. 90° C. A second beaker was filled with 90° C. H₂O also on the hotplate and pumped through the dryer for a couple minutes before and after running solution to prepare/clean the lines of solution. After powering on, the Buchi Mini Spray Dryer B-290 was set to an Inlet temp of 100° C., aspirator of 78%, peristaltic pump speed of 10 (when pumping), atomizer PSI of 45, and a purge rate of 1. Once the system was heated with an outlet temp of approx. 70° C., the 90° C. H₂O was pumped through the lines to help calibrate the system for the solution. After the outlet temp stabilized spraying the H₂O was stopped and spraying of the heated 40% solids solution started. The outlet temperature should remain close to 68° C. Once solution is completed, the heated 90° C. H₂O was again sprayed for another approx. 1 minute to clear the lines of all solution. The material was then removed from the spray dryer and dried in an oven at 80° C. for at least 20 minutes (if needed). Tableting—Once dry, the material was delumped through a mesh screen and bag blended with 1%-2% Magnesium Stearate. If testing for CFU, that material was also bag blended at this point. The material was then dispensed into 800 mg portions to be tableted individually on a Natoli single station NP-RD10A tablet press. Tablets were then compressed at multiple compression forces with a 0.6250 round FFBE tooling. Multiple tablets were tested for tablet characteristics at time of manufacture. Additional tablets were sealed in foil liners with desiccant and left for tablet physicals at desired timeframes.

In certain embodiments, methods of the present disclosure comprise spray congealing (also referred as spray cooling, spray chilling, and melt congealing) an active ingredient and a congealable excipient. Spray congealing generally refers to a process by which a liquid melt or congealable excipient is atomized into a spray of fine droplets of spherical shape inside a cooling chamber. Here, the droplets meet an airstream sufficiently cold to solidify the droplets. The transition of a congealable excipient from a soft or fluid state to a rigid or solid state by cooling is called congealing. Previous studies have shown the use of spray congealing for the preparation of microparticles with the objective of increasing the solubility and dissolution rate of APIs with poor water solubility (Int. J. Pharm. 2009, 381(2), 176-83), obtaining controlled-release dosage forms (Eur. J. Pharm. Bio. 2008, 70, 409-20) and taste masking applications (Chem. Pharm. Bull. 1999, 47(2), 220-25).

Congealable excipients useful in various embodiments of the present disclosure can be selected from the group consisting of DYNASAN® 116, DYNASAN® 118, STEROTEX® GTP, STEROTEX® NF, STEROTEX® K, hydrogenated castor oil, cocoa butter, synthetic wax, microcrystalline wax, paraffin wax, long-chain alcohols, such as stearyl alcohol, cetyl alcohol and polyethylene glycol, ether-substituted cellulosics, such as microcrystalline cellulose, hydroxypropyl cellulose, hydroxypropyl methyl cellulose and ethylcellulose, long-chain fatty acid esters, such as glyceryl monooleate, glyceryl monostearate, glyceryl palmitostearate, polyethoxylated castor oil derivatives, glyceryl dibehenate, triglyceride, mixtures of mono-, di-, and triacyl glycerides, including mixtures of glyceryl mono-, di-, and tribehenate, glyceryl tristearate, glyceryl tripalmitate and hydrogenated vegetable oils, waxes, such as carnauba wax and white and yellow beeswax, carboxylic acids such as stearic acid, benzoic acid, and citric acid.

In certain embodiments, methods of the present disclosure can comprise a drying step. Drying can be performed by any method known to a person of skill in the art. In certain embodiments, a composition can be dried using a desiccant. In another embodiment, a composition can be dried by heating (e.g., using an oven). In yet another embodiment, a composition can be dried by air drying.

Compositions of the present disclosure can be stored for a period (e.g., post compression into a tablet) to allow for post-compression hardening to increase hardness of the dosage form. In certain embodiments, a composition can be stored for a period of about 6 hours, about 12 hours, about 16 hours, about 24 hours, about 36 hours, about 48 hours, or greater than about 48 hours. In certain embodiments, the hardness (kP) per compression (kN) force used to form a solid dosage form is at least about 0.001, at least about 0.002, at least about 0.003, at least about 0.004, at least about 0.005, at least about 0.0075, at least about 0.01, at least about 0.1, at least about 0.25, at most about 0.5, at least about 1.0, at least about 1.5, at least about 2.0, at least about 2.5, at least about 3.0, at least about 3.5, at least about 4.0, at least about 4.5, at least about 5.0, at least about 6, at least about 7, at least about 8, at least about 9, or at least about 10 after less than about 6 hours of storage (e.g., in desiccated conditions, or in the absence of moisture and/or heat). In certain embodiments, the hardness (kP) per compression force (kN) used to form a solid dosage form is at least about 1.0, at least about 1.5, at least about 2.0, at least about 2.5, at least about 3.0, at least about 3.5, at least about 4.0, at least about 4.5, at least about 5.0, at least about 6, at least about 7, at least about 8, at least about 9, or at least about 10 after less than about 12 hours of storage (e.g., in desiccated conditions, or in the absence of moisture and/or heat). In certain embodiments, the hardness per compression force used to form a solid dosage form is at least about 1.0, at least about 1.5, at least about 2.0, at least about 2.5, at least about 3.0, at least about 3.5, at least about 4.0, at least about 4.5, at least about 5.0, at least about 6, at least about 7, at least about 8, at least about 9, or at least about 10 after less than about 16 hours of storage (e.g., in desiccated conditions, or in the absence of moisture and/or heat). In certain embodiments, the hardness (kP) per compression force (kN) used to form a solid dosage form is at least about 1.0, at least about 1.5, at least about 2.0, at least about 2.5, at least about 3.0, at least about 3.5, at least about 4.0, at least about 4.5, at least about 5.0, at least about 6, at least about 7, at least about 8, at least about 9, or at least about 10 after less than about 24 hours of storage (e.g. in desiccated conditions, or in the absence of moisture and/or heat). In certain embodiments, the hardness per compression force used to form a solid dosage form is at least about 1.0, at least about 1.5, at least about 2.0, at least about 2.5, at least about 3.0, at least about 3.5, at least about 4.0, at least about 4.5, at least about 5.0, at least about 6, at least about 7, at least about 8, at least about 9, or at least about 10 after less than about 48 hours of storage (e.g., in desiccated conditions, or in the absence of moisture and/or heat).

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1

In one embodiment using the spray drying process, the powder is manufactured in a Buchi Mini Spray Dryer B-290. A 40% solids solution was made in a 400-600 ml beaker atop a Thermo Fisher Cimarec+SP88857100 Hotplate/Stir plate. The different ratio mixtures of Mannitol/Sorbitol/Maltitol were added to 180 g H₂O while agitating with a stir bar and heating in a covered beaker to approx. 90° C. A second beaker was filled with 90° C. H₂O also on the hotplate and pumped through the dryer for a couple minutes before and after running solution to prepare/clean the lines of solution. After powering on, the Buchi Mini Spray Dryer B-290 was set to an Inlet temp of 100° C., aspirator of 78%, peristaltic pump speed of 10 (when pumping), atomizer PSI of 45, and a purge rate of 1. Once the system was heated with an outlet temp of approx. 70° C., the 90° C. H₂O was pumped through the lines to help calibrate the system for the solution. After the outlet temp stabilized spraying the H₂O was stopped and spraying of the heated 40% solids solution started. The outlet temperature should remain close to 68° C. Once solution is completed, the heated 90° C. H₂O was again sprayed for another approx. 1 minute to clear the lines of all solution. The material was then removed from the spray dryer and dried in an oven at 80° C. for at least 20 minutes (if needed).

Tableting—Once dry, the material was delumped through a mesh screen and bag blended with 1%-2% Magnesium Stearate. The material was then dispensed into 800 mg portions to be tableted individually on a Natoli single station NP-RD10A tablet press. Tablets were then compressed at multiple compression forces with a 0.6250 round FFBE tooling. Tablet hardness was measured immediately after manufacture (t0) and 24 hours (t24) after manufacture using a hardness tester (Dr. Schleuniger Model 6D Tablet Tester). Additional tablets were sealed in foil liners with desiccant and left for tablet physicals at desired timeframes.

TABLE 1
Ratio of polyols in the co-spray dried powder, the initial tablet hardness
(t0) the 24 hour tablet hardness (t24), the % increase in tablet hardness

| Mannitol:sorbitol:maltitol | Compression | T0 hardness | T24 hardness | Hardness |
|---|---|---|---|---|
| ratio | Force (kN) | (Kp) | (Kp) | increase (%) |
| 90:10:0 | 8.7 | 8.5 | 12.7 | 48.8 |
| 80:20:0 | 7.6 | 8.2 | 14.3 | 73.8 |
| 70:30:0 | 7.2 | 8.5 | 13.9 | 63.9 |
| 90:0:10 | 9.7 | 7.7 | 10.6 | 37.7 |
| 80:0:20 | 8.5 | 7.9 | 12.1 | 52.9 |
| 90:5:5 | 9.5 | 8.2 | 10.0 | 22.0 |
| 80:10:10 | 6.7 | 8.8 | 20.0 | 127.3 |
| 70:20:10 | 4.5 | 7.7 | 22.3 | 189.6 |

The results in Table 1 show the influence of formulation on the hardening phenomenon. Without wishing to be bound by any particular theory, a combination of all 3 polyols is required to get the optimum result of a system that compresses remarkably well at low compression forces and gives resultant compacts that have greater than 100% increase in the hardness level. The optimum combination of the 3 polyols seems to be around inclusion of all 3 with the mannitol level being around 70 to 80% and the sorbitol:maltitol level present as either 2:1 or 1:1 in the remaining 20 or 30%. of the powder.

### Example 2

Some of the material manufactured according to the process in Example 1 was investigated for its potential as an excipient used to tablet a blend of material where the active is compression sensitive. The compression sensitive active chosen was the probiotic. Probiotics are known to be inactivated when they are subject to a high compression force being exerted on them. In some embodiments, co-spray dried powder described in example 1 can be used as a means of formulating a probiotic L. acidophilus into a dose form that is subsequently compressed and retains the large majority of the probiotic activity after compression. The approach taken to understand how much of the Probiotic withstood the tabletting process was as follows:

### CFU Testing Using the Spectrometer

### Media Preparation:

Peptone Water—To prepare 532 ml of peptone water, 10.64 g of peptone solution was added to a 1,000 mL graduated cylinder and diluted to 532 mL with purified water. The graduated cylinder was covered with Parafilm and agitated to mix the solution. Once mixed, the solution was dispensed into multiple glass jars, fitted with lose lids, and autoclaved at 121° C. for 15 minutes. After 15 minutes the solution was ready for use.

MRS Broth—To prepare 250 mL MRS Broth solution, 13.75 g of MRS Broth powder was added to a 1,000 mL graduated cylinder and diluted to 250 mL with purified water. Parafilm was then placed over the top of the graduated cylinder and the solution is shaken/mixed thoroughly. An “Eppendorf style” pipette was used to transfer 9 mL of broth to glass tubes to be autoclaved at 121° C. for 15 minutes.

Tablet Preparation—3.82 g of the desired excipient was blended with 0.18 g of L. acidophilus and compressed into 5 tablets. Tablets were produced on a Natoli NP-RD10A single station pressing fitted with 0.6250″ FFBE tooling.

Sample Preparation—Each tablet along with a control of 0.036 mg of L. acidophilous were placed in a whirl-pak. To which, 18 mL of peptone water was added. Each whirl-pak was gently massaged and shaken to break up with tablets and ensure the probiotic was suspended. Once that was completed, the whirl-pak was left to sit for 10 minutes. After 10 minutes, the whirl-pak was shaken to resuspend any particles. 0.2 mL was drawn from the whirl-pak and added to the glass tubes containing MRS broth. Once each tablet and control sample was added to the individual MRS broth containing tubes, the samples were allowed to incubate at 37° C. for four hours.

Testing—After the four hour incubation, the samples were tested for optical density (absorbance) at 600 nm (OD600) using a Thermo Scientific Genesys 10S UV-Vis Spectrophotometer. A blank of MRS broth was used to remove background interference. Samples were transferred to cuvette and the absorbance was measured at 600 nm and compared to the control sample to calculate survivability of the probiotic after compaction

TABLE 2
Summary of results from the above testing

| | Compression | T24 | CFU |
|---|---|---|---|
| Mannitol/sorbitol/maltitol | Force | hardness | retained |
| ratio | (kN) | (kP) | (%) |
| 80/10/10 | 6.7 | 20 | 72.1 |
| 70/20/10 | 4.5 | 22.3 | 98.8 |
| 90/0/10 | 9.7 | 10.6 | 59.2 |
| 80/0/20 | 8.5 | 12.1 | 55.4 |
| 100/0/0(Mannogem XL) | 8.0 | 7.2 | 55.5 |

The results in Table 2 show that optimum combinations of co-spray dried materials shown in example 2 give remarkable results in terms of retaining viability of probiotic materials. It is possible using the materials disclosed herein to compress the blend containing the probiotic powder at a lower compression forces to attain a tablet that hardens within 24 hours to give a robust dose form. The resultant dose form maintain the pressure sensitive Probiotic in its viable state enabling the initial amount of probiotic used in the formula to be significantly reduced as necessary.

### Example 3

In another embodiment some of the material manufactured according to the process in Example 1 was investigated for its potential as an excipient used to tablet a blend of material loaded with silica. Silica is commonly used as a means of adsorbing oils and thus converting liquid systems into solid ones. This is particularly utilised when trying to formulate a poorly soluble lipophilic drug. The drug solubilises readily in oil and this oil can then be adsorbed onto silica and manufactured as a tablet. The limitation of such systems is the poor compressibility of silica with conventional excipients.

Blends of the co-spray dried material made according to the process in Example 1 with a ratio of 85:10:5 mannitol:sorbitol:maltitol was bag blended with 2% magnesium stearate and 15% Syloid XDP 3150. The material was then dispensed into 1000 mg portions to be tableted individually on a Natoli single station NP-RD10A tablet press. Tablets were then compressed at multiple compression forces with a 0.6250 round FFBE tooling.

Table 3 illustrates the capability of the material (manufactured according to the method in Example 1) in a system to sustain large loading of difficult drugs or materials that act as carriers for such for example silica gels such as the Syloid Grades. The hardness of a 1 g tablet containing 83% of the mannitol/sorbitol/maltitol material along with 15% Syloid XDP 3150 and 2% magnesium stearate. The values for initial hardness and hardness after 12 hours are given. The mannitol/sorbitol/maltitol material used in this demonstration contains 85% mannitol along with 10% sorbitol and 5% maltitol (i.e., the 10:5 platform). The 10:5 platform is capable of producing viable tablets, in comparison to other soluble carriers that give failure at or below 5% silica gel loading due to their poor compactibility. In certain embodiments, such a formulation can have similar advantages with difficult to compress actives or with silica gel loaded with oils containing lipophilic drugs such as CBD.

TABLE 3
Tableting performance of 85:10:5 Mannitol:Sorbitol:Maltitol
material with 15% Syloid loading

| | | Average | Average | | Composition |
|---|---|---|---|---|---|
| | Compression | initial | hardness | | of 10:5 in |
| Run | force | hardness | at 12 hrs. | | formulation |
| 1 | 15 kN | 5 kp | 8.7 | kp | 83% |
| 2 | 20 kN | 9 kp | 11.2 | kp | 83% |

### Example 4

In another embodiment using a manufacturing scale spray dry process the powder was manufactured in a spray drier with capacity to produce between 90 and 150 kg of product per hour A spray dried product containing mannitol (80%), maltitol (10%), and sorbitol (10%) was manufactured as follows.

Mannitol, maltitol, and sorbitol were dissolved in hot (>85°) water in a mixing tank, the polyol solution was then pumped at a liquid flow rate between 2 and 7 kg/min from the bottom of the tank to the atomizer located in the drying chamber. Atomizer speed between 5000 and 20000 rpm can be used depending on the particle size of the final product. The inlet air temperature to the atomizing chamber can be varied between 180 and 240° C. giving a resultant outlet air temperature between 70 and 100° C. Similarly drying inlet airflow rates of between 750 and 1600 SCFM were used. The solution is atomized and dried before reaching the bottom of the drying chamber where a fluidized bed further dries the material with fluidization airflows of between 250 and 550 SCFM. Subsequently the materials is discharged into the packaging line.

Processing conditions are chosen to create the desired product in terms of particle size, Loss on Drying and Active Water: Particle size was tested using a granular laser diffraction method on a Microtrac S3500 with 10 psi vacuum. Loss on Drying (L.O.D) was derived using 10 g of material in a Mettler Toledo HR73 Halogen Moisture Analyzer for 10 minutes at 105° C.

Water activity (Aw) was tested using a Aqualab 4TE water activity meter. The bulk and tapped density of the material was tested using a Hosokawa Powder Tester.

TABLE 4
Typical values for particle size distribution, LOD, Aw and density

| Parameter Measured | Results |
|---|---|
| L.O.D (%) | 0.07-0.12 |
| D10 (microns) | 56.5 |
| D50 (microns) | 114.2 |
| D90 (microns) | 192.9 |
| Aw | 0.025-0.120 |
| Bulk density (g/ml) | 0.486 |
| Tapped density (g/ml) | 0.594 |

The material manufactured above with a composition of 80:10:10 (mannitol:sorbitol:maltitol) was then used to manufacture tablets.

The powder was manually blended by shaking contents in bag with lubricant (magnesium stearate) and, as required, griseofulvin as a model drug. 500 mg tablets were made, by compressing the blend at 6.5 to 9 kN depending on the formula being compressed, on a single station press (NP-RD10A) using 0.4375″ FFBE tooling. Tablet hardness was measured immediately after manufacture (t0) and 24 hours (t24) after manufacture using a hardness tester (Dr. Schleuniger Model 6D Tablet Tester)

To compare the properties of the composition of the embodiments the same tablet formulations were manufactured using the same method replacing the powder composition with an alternative soluble directly compressible binder (lactose) in the form of Flowlac 100 or SuperTab. The formulations tested are detailed in Table 5.

TABLE 5

| Ingredient | mg/tablet | % |
|---|---|---|
| Flow Lac 100; SuperTab or | 345 | 69.0 |
| 80:10:10 co spray dried powder | | |
| Griseofulvin | 125 | 25.0 |
| Croscarmellose sodium | 10 | 2.0 |
| Syloid 244FP | 10 | 2.0 |
| Mag. Stearate | 10 | 2.0 |
| Total | 500 | 100 |

To achieve a tablet hardness of 8 Kp for the lactose containing formulations a compression Force of 9.5 kN was required whereas a higher tablet hardness of 10 Kp was achieved with a lower compression force of 6 kN.

Dissolution was performed in accordance to the USP monograph for griseofulvin. The media used was 1000 mL water containing 40 mg/mL sodium lauryl sulfate. Apparatus Type II was used with a mix speed of 75 RPM for 90 minutes.

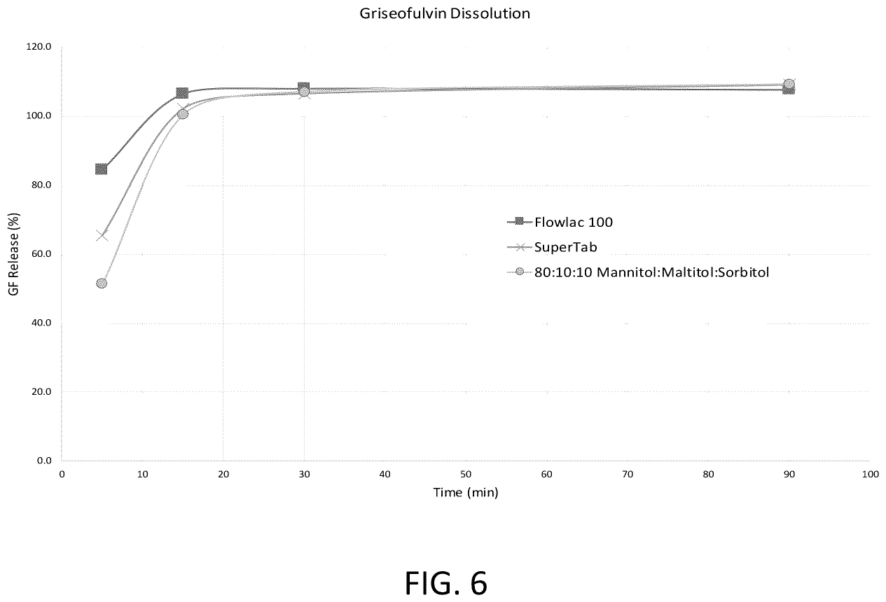

FIG. 6 shows the dissolution of Griseofulvin from tablets made from directly compressible lactose and from 80:10:10 mannitol:sorbitol:maltitol co-spray dried process. It is noticeable that despite the higher hardness of the tablets made with the co-spray dried powder the dissolution performance is still very comparable to the Flowlac tablets easily passing USP monograph limits of 75% release in 90 mins.

The formulations outlined in Table 5 for FlowLac 100 and co-spray dried 80:10:10 mannitol:sorbitol:maltitol were also manufactured as larger blends for compression on a Rotary tablet press as follows:

Blends of 200 g were blended with 2.0% croscarmellose sodium as a disintegrant 2% colloidal silica as a glidant and 2.0% magnesium stearate as a lubricant in an 8 qt v-shell blender. The griseofulvin, croscarmellose, silica and binder were first blended together for 10 minutes. Subsequently the mag stearate was added and blending continued for an additional 5 minutes. For tabletting on a Globe Pharma rotary tablet press (where 1 of the 8 stations had tooling present) FFBE 0.4375″ tooling was used and the range of compression forces used was from 5-15 kN in 5 kN increments.

At each compression force tablets were tested for hardness. The resultant tablet hardness for the above formulas at various compression force at t0 and t24 hours are given in Tables 6 and 7.

TABLE 6
Results for Flowlac100

| | Compression Force (kN) | | |
|---|---|---|---|
| | 5 | 10 | 15 |
| t0 hardness (Kp) | 4.1 | 9.8 | 15.4 |
| t24 hardness (Kp) | 4.2 | 10.1 | 15.5 |
| % increase | 2.0 | 3.5 | 2.1 |

TABLE 7
Results for the 80:10:10 mannitol:sorbitol:maltitol
co spray dried powder

| | Compression Force (kN) | | |
|---|---|---|---|
| | 5 | 10 | 15 |
| t0 hardness (Kp) | 10.6 | 19.1 | 24.0 |
| t24 hardness (Kp) | 12.8 | 22.8 | 28.2 |
| % increase | 20.1 | 19.7 | 17.7 |

### Example 5

In a further embodiment the co-spray dried material produced in Example 4 was compared for its ability to produce robust and hard tablets versus the same ratio of components prepared as a simple blend. The properties and functionality of co-spray dried material produced in Example 4 was compared to a blend of the same components that were not co-spray dried. The material was formulated according to the details in Table 8.

To manufacture these blends and subsequent tablets the following process was used. The materials were blended for 20 minutes in a V-blender with 5% Syloid 244 FP silica followed by an additional 5 minute blend with 2% Lubripharm (sodium stearyl fumarate). 500 mg tablets were then manufactured from each of these 2 blends for comparison on a Globe Pharma GP8 rotary press using 0.4375 round FFBE tooling at 5 kN, 10 kN, and 15 kN compression forces. These tablets were then tested for hardness in a Dr. Schleuniger Pharmatron Model 6D Tablet Tester, friability in an Erweka TA 10 rotary wheel.

TABLE 8
Tablet Formulation Details

| | Co spray dried | |
|---|---|---|
| | 80/10/10 | |
| | mannitol/sorbitol/ | Co blended |
| Ingredient | mannitol binder | material |
| Co spray dried 80/10/10 powder | 279 | 0 |
| mannitol(Mannogem EZ) | 0 | 223.2 |
| Sorbitol (Sorbidex P) | 0 | 27.9 |
| Maltitol (Maltisweet) | 0 | 27.9 |
| Syloid 244FP | 15 | 15 |
| Sodium stearyl fumarate | 6 | 6 |
| (Lubripharm) | | |

TABLE 9
Comparison of results for hardness and friability
of a simple blend of material versus the
co-spray dried material produced in Example 4

| | material | | | | | |
|---|---|---|---|---|---|---|
| | | | | Co-spray dried 80/10/10 | | |
| | | | | mannitol/sorbitol/ | | |
| | blended | | | maltitol material | | |
| | Compression Force (kN) | | | | | |
| | 5 | 10 | 15 | 5 | 10 | 15 |
| t 0 hardness | 2.5 | 6.3 | 10.9 | 6.3 | 13.7 | 19.8 |
| (Kp) | | | | | | |
| t24 hardness | 2.7 | 6.7 | 11.1 | 16.4 | 33.4 | 42.7 |
| (Kp) | | | | | | |
| t 0 Friability | 0.61 | 0.60 | 0.00 | | | |
| (%) | | | | | | |
| t 24 | 1.82 | 0.20 | 0.36 | 0.00 | 0.00 | 0.00 |
| Friability | | | | | | |
| (%) | | | | | | |

The results above show the huge difference in the hardness values of tablets made form the different blends both immediately after manufacture and after 24 hours of case hardening between the blended example and the co-spray dried 80/10/10 mannitol/sorbitol/maltitol material manufactured according to the process in Example 4.

### Example 6

In a further embodiment the co-spray dried material produced in Example 4 was compared for its ability to produce robust and hard tablets to the insoluble binder microcrystalline cellulose that comes in several Grades a standard Grade (Avicel PH102) and a highly compressible grade (Ceolus KG100).

Three separate blends (50 g) were manufactured by blending each binder with 60% acetaminophen and 2.5% Sodium Stearyl Fumarate for lubrication by bag blending for a limited time. 1200 mg tablets were then made using a 0.6875 flat face beveled edge (FFBE) D tooling on a single station Natoli NP-RD10A tablet press. The same compression forces were used in tableting all three blends. These tablets were then tested for hardness at time zero and time 24 hr in a Dr. Schleuniger Pharmatron Model 6D Tablet Tester and friability in an Erweka TA 10 rotary wheel.

TABLE 10

| | Product | | |
|---|---|---|---|
| | Avicel | Asahi | Super Dry |
| | PH-102 | KG-1000 | Binder |
| Initial Hardness (kP) | 2.0 | 8.8 | 5.0 |
| 24 Hour Hardness (kP) | 2.3 | 9.1 | 9.9 |
| Friability (% Friable) | 100.0% | 0.0% | 0.5% |

Table 10 shows the remarkably high tablet hardness after 24 hours for the 80:10:10 (mannitol:sorbitol:maltitol) co spray dried powder containing a high (60%) drug loading (Super Dry Binder in Table 10 is the 80:10:10 mannitol:sorbitol:maltitol co-sprayed dried material). The hardness and friability data after 24 hours are close to the values obtained from the insoluble highly compressible grade of microcrystalline cellulose.

### Example 7

In a further embodiment the co-spray dried material produced in Example 4 along with direct compressible spray dried mannitol was compared as follows:

Blends of 200 g were manufactured containing the poorly compacting API (Acetaminophen, Special Granular from Mallinckrodt mean particle size for the acetaminophen was ˜250 μm.) using either the co-spray dried material produced in Example 2 as the binder or alternatively a spray dried DC mannitol (Pearlitol 200 SD). All formulations were blended with the acetaminophen and 2.5% sodium stearyl fumarate (SSF) as a lubricant in an 8 qt v-shell blender. The acetaminophen, and binder were first blended together for 10 minutes. Subsequently the SSF was added and blending continued for an additional 5 minutes. For the blend with the Pearlitol SD 200 mannitol the acetaminophen loading was 15%, whereas for the 80:10:10 (mannitol:sorbitol:maltitol) co spray dried powder the acetaminophen loading was 20%.

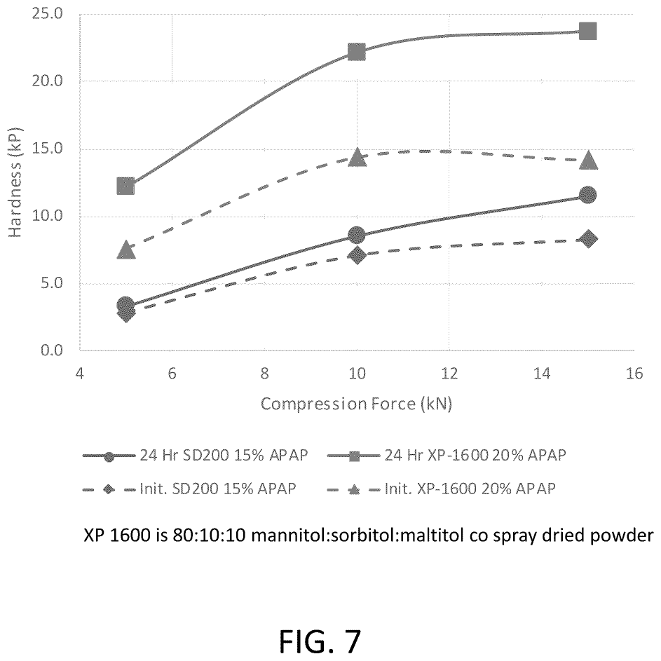

Each blend was tableted on a Globe Pharma rotary tablet press (where 1 of the 8 stations had tooling present) FFBE 0.4375″ tooling was used and the range of compression forces used was from 5-15 kN in 5 kN increments. At each compression force tablets were tested for hardness at t0 and t24 and friability at t24. FIG. 7 shows the hardness values at t0 and t 24 for the formulations described above at various compression forces.

TABLE 11
Friability versus compression force values
for both formulations described above

| | Compression Force (kN) | | |
|---|---|---|---|
| | 5 | 10 | 15 |
| Friability (%) for Pearlitol | 3.04 | 0.28 | 12.80 |
| SD200 with 15% acetaminophen | | | |
| Friability (%) for 80:10:10 | 0.00 | 0.00 | 0.00 |
| (mannitol:sorbitol:maltitol) co | | | |
| spray dried powder | | | |
| with 20% acetaminophen | | | |

The results above show the remarkable improvement in tablet robustness created in the tablets manufactured from the 80:10:10 (mannitol:sorbitol:maltitol) co spray dried powder when compared to standard spray dried mannitol. The results are even more remarkable when one considers that the drug loading in the standard spray dried mannitol samples is 15% versus 20% in the tablets manufactured from the 80:10:10 (mannitol:sorbitol:maltitol) co spray dried powder. Normally it would be expected that tablets containing higher drug loadings would be lower in hardness and higher in friability, however the data clearly shows that the case hardening phenomenon described in this disclosure gives rise to a significant improvement in functional performance.

### Example 8

In a further embodiment the co-spray dried material produced in Example 4 along with other conventional soluble direct compressible binders was were compared as follows:

Blends of either 200 g or 800 g were manufactured containing the poorly compacting API (Acetaminophen, Special Granular from Mallinckrodt mean particle size for the acetaminophen was ˜250 μm.) using various different grades of spray dried DC soluble binder products including mannitol and sorbitol. All formulations were blended with 2.0% croscarmellose sodium as a disintegrant and 2.5% sodium stearyl fumarate (SSF) as a lubricant in an 8 qt v-shell blender. The acetaminophen, croscarmellose and binder were first blended together for 10 minutes. Subsequently the SSF was added and blending continued for an additional 5 minutes. The level of Acetaminophen in the blends was increased in 2.5% increments starting at 12.5%. For tabletting on a Globe Pharma rotary tablet press (where 1 of the 8 stations had tooling present) different tooling was used depending on the tablet size required. Those blends that were compressed with the 0.6250″ FFBE tooling were compressed over a range of compression forces from 15-25 kN in 2.5 kN increments. For the blends compressed with the FFBE 0.4375″ tooling the range of compression forces were used was from 5-15 kN in 2.5 kN increments. Tabletting was undertaken at each acetaminophen weight loading until the tablets produced were not sufficiently robust (friability values were >1%). At each compression force tablets were tested for hardness and friability

TABLE 12
Comparison of the drug loading achievable for each of the different
formulas tested, together with friability and hardness data

| | Drug | | | | | |
|---|---|---|---|---|---|---|
| | Load (%) | | | | | |
| | achievable | Tablet weight | | | | |
| | that meets | for 160 mg | | | | |
| | friability | Acetaminophen | Friability | Comp | Hardness | Hardness |
| Product | of <1% | dose (mg) | (%) | force (kN) | t₀ (Kp) | t₂₄ (Kp) |
| Pearlitol | 12.5 | 1280 | 0.45 | 20 | 14.4 | 16.4 |
| 200SD | | | | | | |
| DC Sorbitol | 15 | 1067 | 0.26 | 25 | 13.8 | 16.4 |
| (Sorbitab | | | | | | |
| SD250) | | | | | | |
| Mannogem XL | 15 | 1067 | 0.22 | 20 | 12.4 | 15.2 |
| 80:10:10 co | 37.5 | 427 | 0.16 | 10 | 5.6 | 8.0 |
| spray dried | | | | | | |
| material | | | | | | |

The data in Table 12 shows a remarkable and surprising ability of the co-spray dried material to enable the incorporation of high levels of acetaminophen (37.5% versus 12.5 to 15%) in a direct compression tablet that has acceptable robustness (friability <1%)) when compared to other soluble binder materials such as sorbitol and mannitol. As such this material has exceptional potential to enable formulators to reduce the amount of any excipient in a given tablet formula to enable much smaller tablets to be produced.

### Example 9

In a further embodiment the co-spray dried material produced in Example 4 along with other conventional direct compressible binders (namely Microcelac, ProSolv HD90, LudiPress, and Avicel HFE) was were compared as follows:

Acyclovir was used as a model drug at 60% drug loading, with acyclovir dose of 200 mg and total tablet weight of 363.50 mg. The blend was prepared using the materials as per Table 13.

TABLE 13

| Ingredients | mg/tablet | Batch quantity (g) |
|---|---|---|
| Acyclovir | 214.75 | 214.75 |
| binder | 127.75 | 127.75 |
| Colloidal silicon dioxide | 3.50 | 3.50 |
| Crospovidone XL 10 | 10.50 | 10.50 |
| Magnesium stearate | 7.00 | 7.00 |
| Total Weight | 363.50 | 363.50 |

The batch was prepared by co-sifting of Acyclovir along with the binder, Crospovidone XL-10 and Colloidal Silicon dioxide via #30 mesh, followed by blending using a V Cone blender (Kalweka), at 18 RPM for 10 minutes. The blend was lubricated with 2% Magnesium stearate pre-sieved through a #60 mesh sieve and blended in the V Cone blender for 5 minutes.

Compaction was undertaken using 11.11 mm flat faced bevelled edge, round shape tableting tools. Tablets were prepared using gravity feed Instrumented tablet press (Pacific SRC10i) set at 15 rpm with compaction force of 4, 8 and 12 kN. The average tablet target weight was 363.50 mg. Tablet hardness was tested using an ERWEKA TBH-125 hardness testing equipment (n=10). Disintegration was tested utilizing a Pharmatest PTZ A2E2 apparatus according to USP Method (n=6) and friability using an ERWEKA iTAR friabilator again according to the USP method adapted for 100 and 300 revolutions to enable a more thorough understanding of tablet robustness. Dissolution for each formulation made was also tested. The dissolution media was 0.01 N HCl, with 900 ml volume in each dissolution flask and the method apparatus USP II at 50 RPM (n=6). The results are set forth in Table 14-18 below:

TABLE 14
Results using co-spray dried material
produced in Example 2 as Binder

| | Compression Force (kN) | | |
|---|---|---|---|
| | 4 | 8 | 12 |
| Mean Hardness (N) | 83.7 | 127.5 | 150.7 |
| Friability at 100 rev | 0.23 | 0.21 | 0.18 |
| (%) | | | |
| Friability at 300 rev | 1.17 | 0.53 | 0.53 |
| Disintegration Time | 51 | 127 | 199 |
| (secs) | | | |

TABLE 15
Results using microcelac as Binder

| | Compression Force (kN) | | |
|---|---|---|---|
| | 4 | 8 | 12 |
| Mean Hardness | 46.5 | 85.2 | 112.1 |
| (N) | | | |
| Friability at 100 | 3.04 | 0.55 | 0.24 |
| rev (%) | | | |
| Friability at 300 | n/d | 3.47 | 1.13 |
| rev | | | |
| Disintegration | 40 | 46 | 51 |
| Time (secs) | | | |

TABLE 16
Results using ProSolv HD90 as a Binder

| | Compression Force (kN) | | |
|---|---|---|---|
| | 4 | 8 | 12 |
| Mean Hardness (N) | 52.7 | 101.8 | 125.4 |
| Friability at 100 rev | 1.41 | 0.17 | 0.11 |
| (%) | | | |
| Friability at 300 rev | 5.10 | 0.93 | 0.30 |
| (%) | | | |
| Disintegration Time | 28 | 26 | 33 |
| (mins) | | | |

TABLE 17
Results using Ludipress as a Binder

| | Compression Force (kN) | | |
|---|---|---|---|
| | 4 | 8 | 12 |
| Mean Hardness (N) | 42.3 | 76.5 | 110.7 |
| Friability at 100 rev | 2.84 | 0.58 | 0.30 |
| (%) | | | |
| Friability at 300 rev | n/d | 2.31 | 0.86 |
| (%) | | | |
| Disintegration Time | 60 | 54 | 69 |
| secs | | | |

TABLE 18
Results using Avicel HFE as a Binder

| | Compression Force | | |
|---|---|---|---|
| | 4 | 8 | 12 |
| Mean Hardness (N) | 57.6 | 103.1 | 130.4 |
| Friability at 100 rev | 0.84 | 0.22 | 0.18 |
| (%) | | | |
| Friability at 300 rev | 3.80 | 0.75 | 0.40 |
| (%) | | | |
| Disintegration Time | 34 | 44 | 22 |
| (secs) | | | |

The results shown in Tables 14 to 21 show that the case hardened excipient according to embodiments of the present disclosure shows remarkable performance when used as a DC soluble binder. The resultant tableted product has superior performance giving robust tablets and lowest friability at the lower compression force applied (4 kN) when compared to all the other binders studied. The resultant product has superior friability under extreme friability conditions making it particular suitable for utilisation in the manufacture of tablets that are subsequently film coated where tablets of unique and difficult shape can be prone to high friability during the coating process.

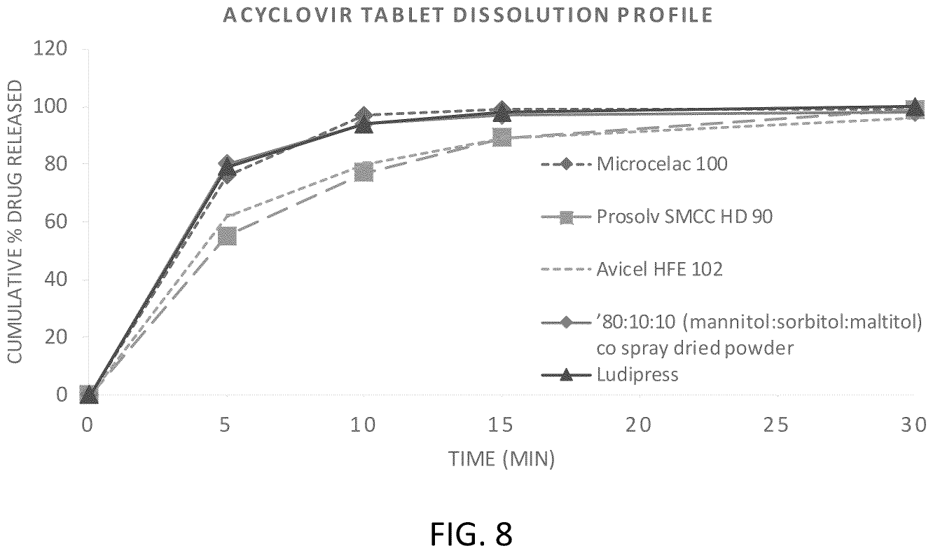

FIG. 8 shows the dissolution release of acyclovir from tablets made with different binder systems. Despite the superior robustness of tablets made with the co-spray dried material produced in Example 4 the dissolution of acyclovir is as fast as any of the other systems.

### Example 10

In a further embodiment the tablets manufactured from the co-spray dried material in Example 4 and compressed at 8 kN compression force as in Example 11 were packed in HDPE bottles and placed in a stability chamber at 40° C. and 75% RH for 1 month. After one month the tablets were removed and tested for hardness, disintegration and dissolution Tablet hardness was tested using an ERWEKA TBH-125 hardness testing equipment (n=10). Disintegration was tested utilizing a Pharmatest PTZ A2E2 apparatus according to USP Method (n=6). The dissolution media was 0.01 N HCl, with 900 ml volume in each dissolution flask and the method apparatus USP II at 50 RPM (n=6).

TABLE 17
Results for hardness and disintegration

| | t0 | t 1 month |
|---|---|---|
| Hardness (N) | 127.5 | 126.6 |
| Disintegration Time (secs) | 126 | 208 |

TABLE 18
Results for dissolution

| | t 0 | t 1 month |
|---|---|---|
| % acyclovir release 5 mins | 80 | 69 |
| % acyclovir release 10 mins | 94 | 94 |
| % acyclovir release 15 mins | 97 | 97 |
| % acyclovir release 30 mins | 98 | 98 |
| % acyclovir release 45 mins | 98 | 98 |

Results show that the tablets produced with acyclovir at 60% drug loading and the co-spray dried binder described in example 4 retain their hardness, DT and dissolution performance after 1 month storage in accelerated conditions.

### Example 11

In a further embodiment ibuprofen was used as a model drug at 70% drug loading, with ibuprofen level of 400 mg per tablet and total tablet weight of 620.00 mg, to determine the tableting properties of the co-spray dried powder manufactured in Example 4. The blend was prepared using the materials as per Table 19.

TABLE 19

| Ingredients | mg/unit | Batch quantity (g) |
|---|---|---|
| Actimask Ibuprofen 92 S | 434.42 | 173.77 |
| Co spray dried powder | 151.18 | 60.47 |
| Colloidal silicon dioxide | 6.40 | 2.56 |
| Crospovidone XL 10 | 19.00 | 7.60 |
| Magnesium stearate | 9.00 | 3.60 |
| Total Weight | 620.00 | 248.00 |

The batch was prepared by co-sifting of Actimask Ibuprofen 92S along with the co spray dried binder material produced in Example 4, Crospovidone XL-10 and Colloidal Silicon dioxide via #30, followed by blending using V cone blender (Make Kalweka model HD 410AC) for 10 minutes. The blend was lubricated with 2% Magnesium stearate (#60 mesh) and blended in a V cone blender for a further 5 minutes.

Compaction was achieved utilizing 12.50 mm flat faced bevelled edge, round shape tableting tooling. Tablets were prepared using gravity feed Instrumented tablet press (make—Pacific SRC 10i) set at 15 rpm with compaction force of 5, 10 and 15 KN. The average tablet target weight was 620 mg. Tablet hardness was tested using ERWEKA TBH-125 and disintegration was tested utilizing a Pharmatest PTZ A2E2. The results are set forth in Table 20 below

TABLE 20

| | Compression Force (kN) | | |
|---|---|---|---|
| | 5 | 10 | 15 |
| Tablet Hardness (N) | 68.3 | 92.5 | 107.9 |
| Friability at 100 | 0.03 | 0.04 | 0.02 |
| revolutions (%) | | | |
| Friability at 300 | 1.61 | 0.86 | 0.85 |
| revolutions (%) | | | |
| Disintegration time | 281 | 467 | 545 |
| (secs) | | | |

The results shown in Table 20 demonstrate a soluble co-spray dried combination of polyols that demonstrate superior compressibility and hardening to be used to formulate a tablet that contains a remarkably high amount of API (70% ibuprofen) using a direct compression process.

### Example 12—Preparation of MUPS Tablets Using Coated Spheres as an Example of Compression Sensitive Active

The use of controlled release particles or pellets, that is, small spheres (150-800 microns in diameter) as a substrate for drug coating and subsequent application of a controlled release polymer is a well-known approach in the field for delivering drugs that are normally administered multiple times daily. Instead of the patient taking 3 or 4 tablets a day the dose can be loaded into one dose form and the drug released in a controlled manner throughout a 24 hour period. Such coated particles or spheres are by their nature small and so need to be combined into a reliable single dose form such as a tablet. Such dose forms or tablets are known as MUPS tablets. One challenge with such coated particles is their sensitivity to compression force as the force needed to compress the tablets can cause significant damage to the rate controlling polymer rendering the dose form unable to meet its design objectives.

By being able to compress such systems at very low compression forces (e.g., 2 versus 10 kN) and still being able to form a robust dose form of sufficient hardness and low friability the formulator would more easily be able to quickly design and manufacture tablet formulations that meet the intended design criteria.

Cetirizine was used as a model drug at 10% drug loading on seal coated Non-pareil seeds (sugar spheres), followed by sustained release coating and compressed with tablet strength of 10 mg and total tablet weight of 500.00 mg, to determine the tableting properties of material manufactured in Example 4 with 80:10:10 mannitol:sorbitol:maltitol. The various steps involved were given below:

### Seal Coating of Sugar Spheres

The Non-pareil seeds were seal coated using hydroxypropyl methyl cellulose (5 cP) at a concentration of 5% w/v, to get the spheres of smooth surface with enough hardness for Cetirizine drug layering. The composition of seal coating solution used is given in Table 21; 30% overage of coating materials was used to compensate the process losses during coating.

TABLE 21

| SL No. | Ingredients | Quantity (g/batch) |
|---|---|---|
| 1 | Sugar spheres | 600.00 |
| 2 | HPMC | 39.00 |
| 3 | PEG 400 | 7.80 |
| 4 | Yellow color | 0.4 |
| 5 | DM Water | 455.00 |

| | |
|---|---|
| Total solids | 647.2 |

The seal coating solution was prepared by heating 150 mL of water at 90° C. followed by addition of HPMC using Magnetic stirrer (Remi 5MLH), at 200 RPM and the mixing was continued until all particles are thoroughly wetted, around 10 Min. The remaining water quantity of water was added slowly to lower the temperature of the dispersion. Further, PEG 400 followed by yellow color was added and the stirring was continued for additional 30 min. The coating solution was kept at ambient condition till it reach the room temperature.

The seal coating of sugar spheres was done by placing 600 g of sugar spheres in a GPCG 1.1, fluid bed coater (bottom spray mode (ACG Capsules), using a Type B plate. The coating process parameters used for the seal coating of the sugar spheres are given in Table 22.

TABLE 22

| Parameters | Actual value | |
|---|---|---|
| Spray nozzle | 0.8 | mm |
| Spray tube | 7 mm (OD) 4 mm (ID) | |
| Air distribution plate | Type B | |
| Inlet temperature | 45-60° | C. |
| Product temperature | 33-36° | C. |
| Air flow | 60-70CFM | |
| Blower speed | 40-42 | |
| Atomization air pressure | 1.2 | bar |
| Spray rate | 3.6 | g/min |
| Curing | 40 min at 40° C. | |
| Initial weight of sugar | 600.0 | g |
| spheres | | |
| Final weight of sugar | 633.6 | g |

| | |
|---|---|
| spheres after seal coating | |
| Percentage coating | 5% |
| D50 of starting spheres | 516 |
| (microns) | |

### Cetirizine Layering on Seal Coated Sugar Spheres

The Cetirizine hydrochloride drug was coated at 10% w/w on seal coated sugar spheres in solution form using the ingredients given in Table 23; 30% overage of drug and coating materials were used to compensate the process losses during coating.

TABLE 23

| | | Quantity |
|---|---|---|
| Sl. No. | Ingredients | (g/batch) |
| 1 | Sugar spheres seal coated | 400 |
| 2 | HPMC (Methocel E15) | 10.4 |
| 3 | Cetirizine HCl IP | 52.0 |
| 4 | Talc | 5.2 |
| 5 | PEG 400 | 2.08 |
| 6 | Red color | 0.39 |
| 7 | DM Water | 585.00 |

| | |
|---|---|
| Total solids (g) | 470.07 g |

The Cetirizine HCl solution was prepared by heating 150 mL of water at 90° C. followed by addition of HPMC using Magnetic stirrer (Remi 5MLH), at 200 RPM and the mixing was continued until all particles are thoroughly wetted, around 10 min. Talc and Red color were dispersed in another 150 mL water using Magnetic stirrer (Remi 5MLH), at 200 RPM for 10 Min. This solution was added along with the HPMC solution, followed by PEG 400 and stirred for an additional 10 min. Cetirizine HCl is accurately weighed for 52 g and dissolved in 150 mL of water separately and is added to the coating solution at 30 to 35° C. temp. The remaining quantity of water was added slowly to lower the temperature of the dispersion. The coating solution was kept at ambient condition till it reach the room temperature. The was passed through #40ASTM mesh.

The Cetirizine HCl drug layering of sugar spheres was done by placing 400 g of seal coated sugar spheres in GPCG 1.1 fluid bed coater, bottom spray mode (ACG Capsules), using Type B plate. The coating process parameters used for Cetirizine HCl drug layering on seal coated sugar spheres are given in Table 24.

TABLE 24

| Parameters | Actual value | |
|---|---|---|
| Spray nozzle | 0.8 | mm |
| Spray tube | 7 mm (OD) 4 mm (ID) | |
| Air distribution plate | Type B | |
| Inlet temperature | 50-60° | C. |
| Product temperature | 35-38° | C. |
| Air flow | 60-65CFM | |
| Blower speed | 35-45 | |
| Atomization air pressure | 1.5-1.8 | bar |
| Spray rate | 2.6 | g/mins |
| Curing | 60 min at 40° C. | |
| Initial weight of seal | 400.0 | g |
| coated sugar spheres | | |
| Final weight of sugar | 458.3 | g |

| spheres after seal coating | |
|---|---|
| Percentage coating | 10.8% |
| D50 of starting spheres | 540 |
| (microns) | |

### Ethyl Cellulose Coating (Surelease E-7)

Ethyl cellulose sustained release polymer was coated onto the Cetirizine layered coated sugar spheres (300 g) in solution form using the ingredients given in Table 25; 30% overage of coating materials were used to compensate the process losses during coating.

TABLE 25

| Sl. No. | Ingredients | Quantity (g/batch) |
|---|---|---|
| 1 | Cetirizine spheres | 300.00 |
| 2 | Ethyl cellulose | 78.0 (19.5 g |
| | (Surelease E-7) | solid) |
| 3 | Erythrosine B (Pigment) | 0.30 |
| 4 | DM Water | 300.00 |

| Total solids | 319.3 g |
|---|---|
| | (total solid weight) |

Ethyl cellulose aqueous dispersion was prepared by dispersing 78 g of Surelease E7 in 300 mL of water under stirring using Magnetic stirrer (Remi 5MLH), at 200 RPM for 10 min. Erythrosine B pigment was dissolved in another 150 mL water using Magnetic stirrer (Remi 5MLH), at 200 RPM for 5 Min. This solution is added to the Ethyl cellulose dispersion and stirred for an additional 10 min.

Ethyl cellulose, sustained release polymer coating on Cetirizine layered sugar spheres was undertaken by placing 300 g of Cetirizine layered sugar spheres in a GPCG 1.1, fluid bed coater bottom spray mode (ACG Capsules), using Type B plate, The coating process parameters used for Ethyl cellulose coating on Cetirizine layered sugar spheres are given in Table 26.

TABLE 26

| Parameters | Actual value | |
|---|---|---|
| Spray nozzle | 0.8 | mm |
| Spray tube | 7 mm (OD) 4 mm (ID) | |
| Air distribution plate | Type B | |
| Inlet temperature | 55-60° | C. |
| Product temperature | 35-38° | C. |
| Air flow | 60-65CFM | |
| Blower speed | 45-55 | |
| Atomization air pressure | 1.8 | |
| Spray rate | 2.9 | g/mins |
| Curing | 60 mins at 40° C. | |
| Initial weight of sugar | 300.0 | g |
| spheres | | |
| Final weight of sugar | 315.3 | g |

| spheres after seal coating | |
|---|---|
| Percentage coating | 5.0% |
| Yield | 99.6% |
| D50 of starting spheres | 551 |
| (microns) | |

### Compression of Cetirizine SR Coated Sugar Spheres Using 80:10:10 Mannitol:Sorbitol:Maltitol

The Sustained release Cetirizine layered pellets produced above were used as a model spheres at 21.37% drug loading, with tablet strength of 10 mg and total tablet weight of 500.0 mg, to determine the properties of tablets containing 80:10:10 mannitol:sorbitol:maltitol and coated spheres (a so called Multi Unit Particulate System MUPS). The blend was prepared using the materials as per Table 27.

TABLE 27

| Sr. No | Ingredients | mg/unit | Batch size (g) |
|---|---|---|---|
| 1 | Cetirizine Pellets | 106.84 | 74.80 |
| 2 | 80:10:10 | 378.16 | 264.71 |
| | mannitol:sorbitol:maltitol | | |
| 3 | Croscarmellose sodium | 10.00 | 7.00 |
| 4 | Magnesium stearate | 5.00 | 3.50 |
| 5 | Total Weight | 500.00 | 350.00 |

The batch was prepared by co-sifting of SR coated Cetirizine spheres along with 80:10:10 mannitol:sorbitol:maltitol, Croscarmellose sodium through a #30 sieve, followed by blending using V cone blender (Make Kalweka model HD 410AC) for 10 minutes. The blend was lubricated with 1.0% Magnesium stearate (#60 mesh) and blended in a V cone blender for 5 minutes.

Compaction involved a 12.0 mm flat faced beveled edges, round shape tableting tools. Tablets were prepared using gravity feed Instrumented tablet press (make—Pacific SRC 10i) set at 15 rpm with compression force of 2.0 KN. The average tablet target weight was 500 mg. Tablet hardness was tested using ERWEKA TBH-125 and disintegration was tested utilizing a Pharmatest PTZ A2E2. Tablet friability was measured on a friabilator according to USP monograph. The results are set forth in Table 28 below:

TABLE 28
Compaction force of 2.0 kN

| | Hardness | Thickness | Diameter | Disintegration |
|---|---|---|---|---|
| Parameter | (N) | (mm) | (mm) | (Seconds) |
| Average | 61 | 3.83 | 12.04 | 115 |
| Std. Deviation | 2.7 | 0.0 | 0.0 | 6.8 |
| Friability (%) | 0.08% w/w | | | |
| (100 rev) | | | | |

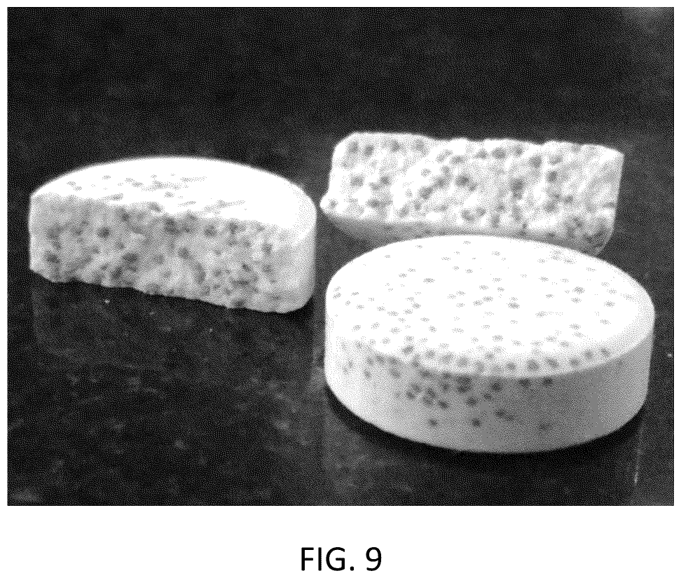

All the tabletting parameters were found to be satisfactory at a low compression force of 2.0 kN. The tablets compressed were of having adequate hardness and lower friability with smooth surface. The picture of compressed tablets is provided in FIG. 9. The MUPS tablets formed are of remarkable robustness given the low compression force of 2 kN used to compress them.

The examples set forth above are provided to give those of ordinary skill in the art a complete disclosure and description of how to make and use the embodiments of the compositions, systems and methods of the present disclosure, and are not intended to limit the scope of what the inventors regard as their invention. Modifications of the above-described modes for carrying out embodiments of the present disclosure that are obvious to persons of skill in the art are intended to be within the scope of the following claims. All patents and publications mentioned in the specification are indicative of the levels of skill of those skilled in the art to which the invention pertains.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

It is to be understood that the methods described herein are not limited to the particular methodology, protocols, subjects, and sequencing techniques described herein and as such can vary. It is also to be understood that the terminology used herein is for the purpose of describing particular embodiments only and is not intended to limit the scope of the methods and compositions described herein, which will be limited only by the appended claims. While some embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and compositions within the scope of these claims and their equivalents be covered thereby.

Several aspects are described with reference to example applications for illustration. Unless otherwise indicated, any embodiment can be combined with any other embodiment. It should be understood that numerous specific details, relationships, and methods are set forth to provide a full understanding of the features described herein. A skilled artisan, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

While some embodiments have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention.

Furthermore, it shall be understood that all aspects of the invention are not limited to the specific depictions, configurations or relative proportions set forth herein which depend upon a variety of conditions and variables. It should be understood that various alternatives to the embodiments of the invention described herein can be employed in practicing the invention. It is therefore contemplated that the invention shall also cover any such alternatives, modifications, variations or equivalents. It is intended that the following claims define the scope of the invention and that methods and compositions within the scope of these claims and their equivalents be covered thereby.

1-22. (canceled)

23. A dose form selected from a chewable, a tablet, a minitablet, a wafer, a compact, a lozenge, a granule, a hard capsule plug, an effervescent, and a ribbon, wherein the dose form comprises an excipient system comprising two or more polyols, and one or more other excipients selected from the group consisting of sodium stearyl fumarate, magnesium stearate, micro crystalline cellulose, dicalcium phosphate, cellulose, hydroxypropyl cellulose, colloidal silica, fumed silica, PEG, talc, flavors, colors, calcium carbonate, cyclodextrin, gelatin, cellulose ethers, sweeteners, stearic acid, citric acid, hydrogenated castor oil, glyceryl monostearate, methylcellulose, polysorbate, titanium dioxide, starch, a super disintegrant, alginate, lactose, maltose, sucrose, glucose, polydextrose, dextrose, and PVP, wherein upon further processing or storage, a hardness value of the dose form increases by at least 50%.

24. The dose form of claim 23, further comprising one or more of an active pharmaceutical ingredient, a nutraceutical ingredient, a veterinary product, a probiotic, a detergent, a pressure sensitive component, and/or a food supplement.

25. The dose form of claim 23, wherein the dose form weighs in the range of about 10 mg to about 4500 mg.

26. (canceled)

27. The dose form of claim 23, wherein the dose form has a moisture content determined by water activity to be about 0.0225 to about 0.4 and/or a moisture content determined by loss on drying or Karl Fischer titration to be about 0.05% to about 5.0%.

28. (canceled)

29. The dose form of claim 23, wherein, upon further processing or storage, the dose form has a moisture content determined by water activity to be about 0.01 to about 0.5 or determined by loss on drying or Karl Fischer titration to be about 0.05% to about 5.0%.

30-31. (canceled)

32. The dose form of claim 23, wherein the dose form has a hardness of about 0.6 kilopond (kP) to about 30 kP and/or a friability of about 0.05% to about 5%.

33. (canceled)

34. The dose form of claim 23, wherein, upon storage or further processing, a hardness of the dose form is between about 1.6 kilopond (kP) and about 50 kP.

35. The dose form of claim 23, wherein the two or more polyols comprise mannitol, sorbitol, and maltitol.

36. The dose form of claim 35, wherein the ratio of mannitol:sorbitol:maltitol is about 80:10:10 by weight or about 70:20:10 by weight.

37. (canceled)

38. The dose form of claim 24, wherein:
the probiotic CFU is in the range of 100% of the intended label claim prior to compaction, which following initial compaction the probiotic CFU count decreases by less than between about 0.1% to about 50%;
the probiotic loading is in the range of between about 0.1% to about 50% by weight of the finished dosage form, wherein the CFU count following further processing or storage is about the same; or
upon further processing or storage the hardness of the dose form increases by at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, or at least about 250%, at least about 300%, at least about 350%, or at least about 400%.

39-41. (canceled)

42. The dose form of claim 24, wherein the pressure sensitive component is a controlled release or taste masked pellet, granule, core, or particle containing an active pharmaceutical, an ingredient, a nutraceutical ingredient, a veterinary ingredient, a probiotic, a vitamin, a detergent or a food supplement; or wherein the active pharmaceutical ingredient is aspirin, paracetamol, ibuprofen, diclofenac, naproxen, guaiphenesin, loratadine, dextromethorphan, pseudoephedrine, famotidine, cetirizine, nicotine, amlodipine, sildenafil, ondansetron, loperamide, tadalafil, benzodiazepine, clopidogrel, fenofibrate, cannabidiol, isosorbide mononitrate, levothyroxine, lisinopril, losartan, lovastatin, metformin, montelukast, omeprazole, paroxetine, prednisolone, simvastatin, venlafaxine, or zolpidem.

43-47. (canceled)

48. A solid dosage form, comprising:
(i) two or more polyols co-processed to form a homogeneous material, wherein the two or more polyols are independently selected from the group consisting of mannitol, sorbitol, maltitol, xylitol, erythritol, hydrogenated starch hydrolysates, isomalt, and lactitol, wherein the two or more polyols comprises a first polyol present in an amount of about 5 wt % to about 25 wt %, and a second polyol present in an amount of 5 wt % to 25 wt %; and
(ii) one or more compression-sensitive or pressure-sensitive active ingredients,
wherein (1) a hardness (in kiloponds, kP) of the solid dosage form per compression force (in kilonewtons, kN) used to form the solid dosage form is at least about 2.0 after less than about 24 hours of storage, and/or (2) the solid dosage form comprises a first hardness at time t₀ and a second hardness at time t₁ that is at least about 50% greater than the first hardness, wherein the time t₁ is about 4 hours, about 8 hours, about 12 hours, about 16 hours, about 20 hours, or about 24 hours after the time t₀.

49. The solid dosage form of claim 48, wherein the solid dosage form comprises a water activity (Aw) of less than about 0.7, less than about 0.6, less than about 0.5, less than about 0.4, less than about 0.3, less than about 0.2, less than about 0.1, less than about 0.05, or less than about 0.01.

50. The solid dosage form of claim 48, wherein the hardness (in kP) of the solid dosage form per compression force (in kN) used to form the solid dosage form is at least about at least about 1.0 kP/kN, at least about 1.5 kP/kN, at least about 2.0 kP/kN, at least about 2.5 kP/kN, at least about 3.0 kP/kN, at least about 3.5 kP/kN, at least about 4.0 kP/kN, at least about 4.5 kP/kN, at least about 5.0 kP/kN, at least about 6 kP/kN, at least about 7 kP/kN, at least about 8 kP/kN, at least about 9 kP/kN, or at least about 10 kP/kN after less than about 24 hours, less than about 12 hours, or less than about 6 hours of storage.

51-52. (canceled)

53. The solid dosage form of claim 48, wherein the one or more compression-sensitive or pressure-sensitive active ingredients is independently selected from the group consisting of an active pharmaceutical ingredient, a nutraceutical ingredient, a veterinary product, a probiotic, a detergent, and a food supplement.

54. (canceled)

55. The solid dosage form of claim 48, wherein the ratio of the first polyol to the second polyol is about 1:1, about 2:1, or about 1:2.

56-62. (canceled)

63. The solid dosage form of claim 48, wherein the solid dosage form is a chewable, a swallow tablet, a wafer, a compact, a ribbon, an orally disintegrating tablet (ODT), a lozenge, a fast melt, a bi-layer, a tri-layer, a minitablet, a granule, a hard capsule plug, or an effervescent.

64. The solid dosage form of claim 48, further comprising one or more excipients independently selected from the group consisting of sodium stearyl fumarate, magnesium stearate, micro crystalline cellulose, starch, a super disintegrant, alginate, lactose, maltose, sucrose, glucose, polydextrose, dextrose, and PVP.

65. The solid dosage form of claim 48, wherein the two or more polyols comprise mannitol, sorbitol, and maltitol.

66. The solid dosage form of claim 65, wherein the ratio of mannitol:sorbitol:maltitol is about 80:10:10 by weight or about 70:20:10 by weight.

67-96. (canceled)
